(19) United States
(12) Patent Application Publication
(10) Pub. No.: US 2002/0150958 A1
(43) Pub. Date: Oct. 17, 2002

(54) Methods for identifying substances for treating inflammatory conditions

(76) Inventors: Birgit Jung, Schwabenheim (DE); Stefan Mueller, Muenchen (DE); Norbert Kraut, Wien (AT)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/029,654

(22) Filed: Dec. 21, 2001

Related U.S. Application Data
(60) Provisional application No. 60/257,878, filed on Dec. 22, 2000.

(51) Int. Cl. C12Q 1/48; C12Q 1/32

(52) U.S. Cl. 435/15; 435/26

(57) ABSTRACT

The present invention relates to proteins involved in inflammatory processes and the modulation of the function of such a protein in order to positively influence inflammatory diseases.

### RELATED APPLICATION

The benefit of prior United States provisional application No. 60/257,878, filed Dec. 22, 2000 is hereby claimed.

### BACKGROUND

The present invention belongs to the field of modulation of inflammatory processes, in particular of chronic inflammatory airway diseases, in which macrophages play an important role. The inflammatory processes can be modulated according to the invention by influencing the biological activity of a protein which is identified to be involved in the inflammatory process.

An example of a chronic inflammatory airway disease, in which macrophages play an important role is chronic bronchitis (CB). CB may occur with or without airflow limitation and includes chronic obstructive pulmonary disease (COPD). CB is a complex disease encompassing symptoms of several disorders: chronic bronchitis which is characterized by cough and mucus hypersecretion, small airway disease, including inflammation and peribronchial fibrosis, emphysema, and airflow limitation. CB is characterized by an accelerated and irreversible decline of lung function. The major risk factor for developing CB is continuous cigarette smoking. Since only about 20% of all smokers are inflicted with CB, a genetic predisposition is also likely to contribute to the disease.

The initial events in the early onset of CB are inflammatory, affecting small and large airways. An irritation caused by cigarette smoking attracts macrophages and neutrophils the number of which is increased in the sputum of smokers. Perpetual smoking leads to an ongoing inflammatory response in the lung by releasing mediators from macrophages, neutrophils and epithelial cells that recruit inflammatory cells to sites of the injury. So far there is no therapy available to reverse the course of CB. Smoking cessation may reduce the decline of lung function.

Only a few drugs are known to date to provide some relief for patients. Long-lasting β2-agonists and anticholinergics are applied to achieve a transient bronchodilation. A variety of antagonists for inflammatory events are under investigation, for example, LTB₄-inhibitors.

There is a continuous need to provide drugs for treating chronic inflammatory airway diseases. Chronic inflammatory airway diseases can be attributed to activated inflammatory immune cells, e.g. macrophages. There is therefore a need for drugs modulating the function of macrophages in order to eliminate a source of inflammatory processes.

### SUMMARY OF THE INVENTION

The present invention relates to methods for determining whether a substance is an activator or an inhibitor of a function of a protein comprising: (a) contacting the protein with a substance to be tested, wherein the protein is selected from the group consisting of: MIF, DAD1, ARL4, GNS, Transglutaminase 2, Stearyl-CoA-Desaturase and UDP-Glucose Ceramide Glycosyltransferase, or mutants, variants, and fragments thereof; and (b) measuring whether the function is inhibited or activated. The invention encompasses measuring such functions directly or indirectly, and using a cellular or cell-free system. The methods further encompass using mammalian or human protein.

The invention also relates to methods for determining an expression level of a protein comprising: (a) determining the level of the protein in a hyperactivated macrophage, wherein the protein is selected from the group consisting of: MIF, DAD1, ARL4, GNS, Transglutaminase 2, Stearyl-CoA-Desaturase and UDP-Glucose Ceramide Glycosyltransferase; (b) determining the level of the protein in a non-hyperactivated macrophage, wherein the protein is selected from the group consisting of: MIF, DAD1, ARL4, GNS, Transglutaminase 2, Stearyl-CoA-Desaturase and UDP-Glucose Ceramide Glycosyltransferase; and (c) comparing the level of the protein expressed in step (a) to the level of the protein expressed in step (b), wherein a difference in levels indicates a differentially expressed protein.

The present invention also relates to methods for diagnosing or monitoring a chronic inflammatory airway disease comprising: (a) determining the level of the protein in a hyperactivated macrophage, wherein the protein is selected from the group consisting of: MIF, DAD1, ARL4, GNS, Transglutaminase 2, Stearyl-CoA-Desaturase and UDP-Glucose Ceramide Glycosyltransferase; (b) determining the level of the protein in a non-hyperactivated macrophage, wherein the protein is selected from the group consisting of: MIF, DAD1, ARL4, GNS, Transglutaminase 2, Stearyl-CoA-Desaturase and UDP-Glucose Ceramide Glycosyltransferase; and (c) comparing the level of the protein expressed in step (a) to the level of the protein expressed in step (b), wherein a difference in levels indicates a differentially expressed protein. The method further encompasses diagnosing or monitoring a chronic inflammatory airway disease wherein the disease is selected from the group consisting of: CB and COPD.

The present invention also relates to methods for treating a chronic inflammatory airway disease comprising: administering to a subject in need of such treatment an effective amount of a pharmaceutical composition comprising at least one substance determined to be an activator or an inhibitor of a protein selected from the group consisting of: MIF, DAD1, ARL4, GNS, Transglutaminase 2, Stearyl-CoA-Desaturase and UDP-Glucose Ceramide Glycosyltransferase. Such substances may be determined to be activators or inhibitors using the methods of the invention. Preferably, the subject is a mammal, more preferably a human. Preferably, the chronic inflammatory airway disease is selected from the group consisting of: CB and COPD.

The present invention also relates to methods for selectively modulating a protein selected from the group consisting of MIF, DAD1, ARL4, GNS, Transglutaminase 2, Stearyl-CoA-Desaturase and UDP-Glucose Ceramide Glycosyltransferase in a macrophage, comprising administering a substance determined to be an activator or an inhibitor of a protein selected from the group consisting of MIF, DAD1, ARL4, GNS, Transglutaminase 2, Stearyl-CoA-Desaturase and UDP-Glucose Ceramide Glycosyltransferase. The methods further encompass wherein the macrophage is involved in a chronic inflammatory airway disease preferably selected from the group consisting of: CB and COPD.

The present invention also relates to substances determined to be activators or inhibitors of a protein selected from the group consisting of: MIF, DAD1, ARL4, GNS, Transglutaminase 2, Stearyl-CoA-Desaturase and UDP-Glucose Ceramide Glycosyltransferase. Such substances of the invention may be useful for treating a chronic inflammatory airway disease, preferably selected from the group consisting of: CB and COPD.

The invention also encompasses pharmaceutical compositions of such substances.

### DESCRIPTION OF THE INVENTION

In the present invention it was found that macrophages involved in an inflammatory process, particularly in a chronic inflammatory airway disease, more particularly in chronic bronchitis or COPD, show a pattern of differentially expressed nucleic acid sequence and protein expression which differs from the pattern of gene expression of macrophages from healthy donors or donors in an irritated state, which latter do contain macrophages in an activated state. Therefore, macrophages show different activation levels under different inflammatory conditions. For example, it is shown in the present invention that macrophages involved in an inflammatory process in COPD smokers show different gene expression pattern than macrophages from healthy smokers, indicating that in COPD smokers macrophages are in a different, hereinafter named “hyperactivated” or “hyperactive” state. The present invention provides for the inhibition of the hyperactivation or the reduction of the hyperactive state of a macrophage by the identification of substances which modulate a protein selected from the group consisting of MIF (Calandra, T. et al. (1994) J. Exp. Med. 179, 1985-1902; Bernhagen, J. et al. (1998) J. Mol. Med. 76, 151-161; Calandra, T. et al. (2000) Nat. Med. 6, 164-170), DAD1 (Nakashima, T. et al. (1993) Mol. Cell. Biol. 13, 6367-6374; Kelleher, D., and Gilmore, R. (1997) Proc. Natl. Acad. Sci. U.S.A. 94, 4994-4999), ARL4 (Jacobs, S. et al. (1999) FEBS Lett. 456, 384-388), GNS (Kresse, H. et al. (1980) Proc. Natl. Acad. Sci. U.S.A. 77, 6822-6826), Transglutaminase 2, (Folk, J. E. (1980) Annu. Rev. Biochem. 49, 517-531; Lu, S. et al. (1995) J. Biol. Chem. 270, 9748-9756). Stearyl-CoA-Desaturase (Enoch, H. G. et al. (1976) J. Biol. Chem. 251, 5095-5103) and UDP-Glucose Ceramide Glycosyltransferase (Basu, S. et al. (1968) J. Biol. Chem. 243, 5802-5807; Ichikawa, S. et al. (1996) Proc. Natl. Acad. Sci. U.S.A. 93, 4638-4643), all depicted in the Sequence Listing hereinafter, involved in the hyperactivation or maintaining the hyperactive state of a macrophage.

The term “chronic inflammatory airway disease” as used hereinafter includes but is not limited to, Chronic Bronchitis (CB) and Chronic Obstructive Pulmonary Disease (COPD). The preferred meaning of the term “chronic inflammatory airway disease” is CB and COPD, the more preferred meaning is CB or COPD.

The invention is based on the identification of a nucleic acid sequence differentially expressed in a hyperactivated macrophage compared to a macrophage which is not hyperactivated. Such a nucleic acid sequence encodes a protein selected from the group consisting of: MIF, DAD1, ARL4, GNS, Transglutaminase 2, Stearyl-CoA-Desaturase and UDP-Glucose Ceramide Glycosyltransferase, which protein is involved in the hyperactivation or maintenance of the hyperactive state of a macrophage involved in an inflammatory process, preferably in a chronic inflammatory airway disease. Such differentially expressed nucleic acid sequence or protein encoded by such nucleic acid sequence is also referred to hereinafter as differentially expressed nucleic acid sequence or protein of the invention, respectively. In particular, the present invention teaches a link between phenotypic changes in macrophages due to differentially expressed nucleic acid sequence and protein expression pattern and involvement of macrophages in inflammatory processes and, thus, provides a basis for a variety of applications. For example, the present invention provides a method and a test system for determining the expression level of a macrophage protein of the invention or differentially expressed nucleic acid sequence of the invention and thereby provides e.g. for methods for diagnosis or monitoring of inflammatory processes with involvement of hyperactivated macrophages in mammalian, preferably human beings, especially such beings suffering from an inflammatory process, preferably in a chronic inflammatory airway disease. The invention also relates to a method for identifying a substance by means of a differentially expressed nucleic acid sequence or protein of the invention, which substance modulates, i.e. acts as an inhibitor or activator of the said differentially expressed nucleic acid sequence or protein of the invention and thereby positively influences chronic inflammatory processes by inhibition of the hyperactivation or reduction of the hyperactive state of macrophages, and thereby allows treatment of mammals, preferably human beings, suffering from a said disease. The invention also relates to a method for selectively modulating such a differentially expressed nucleic acid sequence or protein of the invention in a macrophage comprising administering a substance determined to be a modulator of said protein or differentially expressed nucleic acid sequence. The present invention includes the use of said substances for treating beings in need of a treatment for an inflammatory process, preferably a chronic inflammatory airway disease.

In the present invention in a first step a differentially expressed nucleic acid sequence of the invention is identified which has a different expression pattern in a hyperactivated macrophage compared to a macrophage which is not hyperactivated. For the sake of conciseness, this description deals particularly with investigation of macrophages involved in COPD; however, equivalent results may be obtained with samples from subjects suffering from other chronic inflammatory airway diseases, e.g. other chronic bronchitis symptoms. The investigation of the different expression pattern leads to the identification of a series of differentially expressed nucleic acid sequences expressed in dependency on the activation state of a macrophage involved in an inflammatory process, as exemplified in the Examples hereinbelow.

Briefly, such a differentially expressed nucleic acid sequence of the invention is identified by comparative expression profiling experiments using a cell or cellular extract from a hyperactivated macrophage, i.e. for example from the site of inflammation in COPD and from the corresponding site of control being not suffering from said disease, however, suffering under the same irritating condition such as cigarette smoke exposure.

In a second step, the proteins are identified which are encoded by the differentially expressed nucleic acid sequence, i.e. proteins playing a role in mediating the hyperactivation or in maintaining the hyperactivated state. A group of differentially expressed nucleic acid sequences of the invention can be identified to encode a protein which is selected from the group consisting of: MIF, DAD1, ARL4, GNS, Transglutaminase 2, Stearyl-CoA-Desaturase and UDP-Glucose Ceramide Glycosyltransferase. A said protein is involved in the hyperactivation or maintenance of the hyperactive state which is characterized in that it is expressed in a macrophage that is hyperactivated according to the invention at a lower or higher level than the control level in a macrophage which is not hyperactivated.

Accordingly, the invention concerns a protein selected from the group consisting of: MIF, DAD1, ARL4, GNS, Transglutaminase 2, Stearyl-CoA-Desaturase and UDP-Glucose Ceramide Glycosyltransferase. A protein selected from the said group is hereinafter also named a protein of the invention. The said proteins of the invention are depicted hereinafter in the Sequence Listing.

The biological activity of MIF (SEQ ID NOs:1, 2) according to the present invention, i.e. mediating the involvement of a macrophage in an inflammatory process according to the invention, e.g. by inhibition of macrophage migration, is dependent, for example, on counteracting suppressive effects of glucocorticoids and/or on another MIF functio, including but not limited to, induction of inflammatory response to invasion of bacteria or any other function of MIF relevant for its biological activity according to the invention.

The invention also concerns a functional equivalent, derivative, variant, mutant or fragment of MIF. Functional in this context means having a function of the MIF that is involved in its biological activity according to the invention.

The biological activity of DAD1 (SEQ ID NOs:3, 4) according to the present invention, i.e. mediating the involvement of a macrophage in an inflammatory process according to the invention, is dependent, for example, on binding to an oligosaccharyltransferase complex and/or on any other DAD1 function relevant for its biological activity according to the invention.

The invention also concerns a functional equivalent, derivative, variant, mutant or fragment of DAD1. Functional in this context means having a function of DAD1 that is involved in its biological activity according to the invention.

The biological activity of ARL4 (SEQ ID NOs:5, 6) according to the present invention, i.e. mediating the involvement of a macrophage in an inflammatory process according to the invention, is dependent, for example, on interaction with proteins involved in vesicular and membrane trafficking and/or on any other ARL4 function relevant for its biological activity according to the invention.

The invention also concerns a functional equivalent, derivative, variant, mutant or fragment of ARL4. Functional in this context means having a function of ARL4 that is involved in its biological activity according to the invention.

The biological activity of GNS (SEQ ID NOs:7, 8) according to the present invention, i.e. mediating the involvement of a macrophage in an inflammatory process according to the invention, is dependent, for example, on binding and/or recognizing a substrate, e.g. heparan and/or on its hydrolytic activity and/or on any other GNS function relevant for its biological activity according to the invention.

The invention also concerns a functional equivalent, derivative, variant, mutant or fragment of GNS. Functional in this context means having a function of GNS that is involved in its biological activity according to the invention.

The biological activity of Transglutaminase 2 (SEQ ID NOs:9, 10) according to the present invention, i.e. mediating the involvement of a macrophage in an inflammatory process according to the invention, is dependent, for example, on formation of (γ-glutamyl) lysine isopeptide bonds and/or on any other Transglutaminase 2 function, e.g. substrate recognition, relevant for its biological activity according to the invention.

The invention also concerns a functional equivalent, derivative, variant, mutant or fragment of Transglutaminase 2. Functional in this context means having a function of Transglutaminase 2 that is involved in its biological activity according to the invention.

The biological activity of Stearyl-CoA-Desaturase (SEQ ID NOs:11, 12) according to the present invention, i.e. mediating the involvement of a macrophage in an inflammatory process according to the invention, is dependent, for example, on binding to a substrate, e.g. palmitoyl-CoA and/or stearyl-CoA and/ or on its oxidative activity and/or on any other Stearyl-CoA-Desaturase function, e.g. substrate recognition, relevant for its biological activity according to the invention.

The invention also concerns a functional equivalent, derivative, variant, mutant or fragment of Stearyl-CoA-Desaturase. Functional in this context means having a function of Stearyl-CoA-Desaturase that is involved in its biological activity according to the invention.

The biological activity of UDP-Glucose Ceramide Glycosyltransferase (SEQ ID NOs:13, 14) according to the present invention, Le. mediating the involvement of a macrophage in an inflammatory process according to the invention, is dependent, for example, on binding to a substrate, e.g. UDP-glucose and/or ceramide and/ or on its transferring activity and/or on any other UDP-Glucose Ceramide Glycosyltransferase function, e.g. substrate recognition, relevant for its biological activity according to the invention.

The invention also concerns a functional equivalent, derivative, variant, mutant or fragment of UDP-Glucose Ceramide Glycosyltransferase. Functional in this context means having a function of UDP-Glucose Ceramide Glycosyltransferase that is involved in its biological activity according to the invention.

According to the present invention, the biological activity of a protein selected from the group consisting of: MIF, DAD1, ARL4, GNS, Transglutaminase 2, Stearyl-CoA-Desaturase and UDP-Glucose Ceramide Glycosyltransferase, if expressed at a lower level than the control level, is preferably activated in order to inhibit hyperactivation or reduce a hyperactivated state of a macrophage, and if expressed at a higher level than the control level, is preferably inhibited in order to inhibit hyperactivation or reduce a hyperactivated state of a macrophage.

In one embodiment, the present invention concerns a test method for determining whether a substance is an activator or inhibitor of a protein selected from the group consisting of: MIF, DAD1, ARL4, GNS, Transglutaminase 2, Stearyl-CoA-Desaturase and UDP-Glucose Ceramide Glycosyltransferase. Since such a protein is involved in a chronic inflammatory airway disease and plays a role in mediating inflammation, a substance modulating the biological activity of such a protein can be used for treating a chronic inflammatory airway disease or can be used as a lead compound for optimization of the function of the substance in a way that the optimized substance is suitable for treating chronic inflammatory airway diseases. For performing a method of the invention, a test system according to the invention can be used.

The present invention also concerns a test system for determining whether a substance is an activator or an inhibitor of a protein selected from the group consisting of: MIF, DAD1, ARL4, GNS, Transglutaminase 2, Stearyl-CoA-Desaturase and UDP-Glucose Ceramide Glycosyltransferase. A test system useful for performing a method of the invention comprises a cellular or a cell-free system. For example, one embodiment of the invention concerns a test system that is designed in a way to allow the testing of substances acting on the expression level of the differentially expressed nucleic acid sequence e.g. using expression of a reporter-gene, e.g. luciferase gene or the like, as a measurable readout. Another embodiment of the invention concerns a test system that is designed in a way to allow the testing of substances directly interacting with a respective function of a protein of the invention or interfering with the respective activation of a function of a protein of the invention by a natural or an artificial but appropriate activator of the respective protein selected from the group consisting of: MIF, DAD1, ARL4, GNS, Transglutaminase 2, Stearyl-CoA-Desaturase and UDP-Glucose Ceramide Glycosyltransferase, e.g. an appropriate kinase or the like.

A test system according to the invention comprises a protein selected from the group consisting of: MIF, DAD1, ARL4, GNS, Transglutaminase 2, Stearyl-CoA-Desaturase and UDP-Glucose Ceramide Glycosyltransferase, or a functional equivalent, derivative, variant, mutant or fragment of a said protein of the invention, a nucleic acid encoding a said protein or encoding a functional equivalent, derivative, variant, mutant or fragment of a said protein of the invention and/or regulatory elements, wherein a functional equivalent, derivative, variant, mutant or fragment of a said protein of the invention or a nucleic acid encoding a said protein or a functional equivalent, derivative, variant, mutant or fragment of a said protein of the invention is able to interact with a substance which should be tested in a way that direct interaction leads to a measurable read-out indicative of the change of a respective biological activity of a said protein according to the invention and /or of the change of expression of a said protein of the invention.

A test system of the invention comprises, for example, elements well known in the art. For example, cell-free systems may include but are not limited to, a said protein or a functional equivalent, derivative, variant, mutant or fragment of a said protein of the invention, a nucleic acid encoding a said protein or encoding a functional equivalent, derivative, variant, mutant or fragment of a said protein of the invention in soluble or bound form or in cellular compartments or vesicles. Suitable cellular systems include, for example, a suitable prokaryotic cell or eukaryotic cell, e.g. such cell comprising a said protein of the invention or a functional equivalent, derivative, variant, mutant or fragment of a said protein of the invention, a nucleic acid encoding a said protein or encoding a functional equivalent, derivative, variant, mutant or fragment of a said protein of the invention (Tsuchiya, S. et al. (1980) Int. J. Cancer 26, 171-176; Ziegler-Heitbrock, H. W. et al. (1988) Int. J. Cancer 41, 456-461). A cell suitable for use in a said test system of the invention may be obtained by recombinant techniques, e.g. after transformation or transfection with a recombinant vector suitable for expression of a desired protein of the invention or functional equivalent, derivative, variant, mutant or fragment of a said protein of the invention, or may e.g. be a cell line or a cell isolated from a natural source expressing a desired protein of the invention or functional equivalent, derivative, variant, mutant or fragment of a said protein. A test system of the invention may include a natural or artificial ligand of the protein selected from the group consisting of: MIF, DAD1, ARL4, GNS, Transglutaminase 2, Stearyl-CoA-Desaturase and UDP-Glucose Ceramide Glycosyltransferase if desirable or necessary for testing whether a substance of interest is an inhibitor or activator of a said protein of the invention.

A test method according to the invention comprises measuring a read-out, e.g. a phenotypic change in the test system, for example, if a cellular system is used, a phenotypic change of the cell. Such change may be a change in a naturally occurring or artificial response, e.g. a reporter gene expression of the cell to a protein selected from the group consisting of: MIF, DAD1, ARL4, GNS, Transglutaminase 2, Stearyl-CoA-Desaturase and UDP-Glucose Ceramide Glycosyltransferase activation or inhibition, e.g. as detailed in the Examples hereinbelow.

A test method according to the invention can on the one hand be useful for high throughput testing suitable for determining whether a substance is an inhibitor or activator of the invention, but also e.g. for secondary testing or validation of a hit or lead substance identified in high throughput testing.

The present invention also concerns a substance identified in a method according to the invention to be an inhibitor or activator of a protein selected from the group consisting of: MIF, DAD1, ARL4, GNS, Transglutaminase 2, Stearyl-CoA-Desaturase and UDP-Glucose Ceramide Glycosyltransferase. A substance of the present invention is any compound which is capable of activating or preferably inhibiting a function of a protein selected from the group consisting of: MIF, DAD1, ARL4, GNS, Transglutaminase 2, Stearyl-CoA-Desaturase and UDP-Glucose Ceramide Glycosyltransferase. An example of a way to activate or inhibit a function of a protein selected from the group consisting of: MIF, DAD1, ARL4, GNS, Transglutaminase 2, Stearyl-CoA-Desaturase and UDP-Glucose Ceramide Glycosyltransferase is by influencing the expression level of a said protein selected from the group consisting of: MIF, DAD1, ARL4, GNS, Transglutaminase 2, Stearyl-CoA-Desaturase and UDP-Glucose Ceramide Glycosyltransferase. Another example of a way to activate or inhibit a function of a protein selected from the group consisting of: MIF, DAD1, ARL4, GNS, Transglutaminase 2, Stearyl-CoA-Desaturase and UDP-Glucose Ceramide Glycosyltransferase is to apply a substance which directly binds a protein selected from the group consisting of: MIF, DAD1, ARL4, GNS, Transglutaminase 2, Stearyl-CoA-Desaturase and UDP-Glucose Ceramide Glycosyltransferase and thereby activating or blocking functional domains of a said protein of the invention, which can be done reversibly or irreversibly, depending on the nature of the substance applied.

Accordingly, a substance useful for activating or inhibiting biological activity of a protein selected from the group consisting of: MIF, DAD1, ARL4, GNS, Transglutaminase 2, Stearyl-CoA-Desaturase and UDP-Glucose Ceramide Glycosyltransferase includes a substance acting on the expression of a differentially expressed nucleic acid sequence, for example a nucleic acid fragment hybridizing with the corresponding gene or regulatory sequence and thereby influencing gene expression, or a substance acting on a protein selected from the group consisting of: MIF, DAD1, ARL4, GNS, Transglutaminase 2, Stearyl-CoA-Desaturase and UDP-Glucose Ceramide Glycosyltransferase itself or on its activation or inhibition by other naturally occurring cellular components, e.g. another protein acting enzymatically on a said protein of the invention, e.g. a protein kinase.

Therefore, the invention concerns, for example, a substance which is a nucleic acid sequence coding for a protein of the invention, or a fragment, derivative, mutant or variant of such a nucleic acid sequence, which nucleic acid sequence or a fragment, derivative, mutant or variant thereof is capable of influencing the gene expression level, e.g. a nucleic acid molecule suitable as antisense nucleic acid, ribozyme, or for triple helix formation.

The invention also concerns a substance which is e.g. an antibody or an organic or inorganic compound which directly binds to or interferes with the activation of a protein selected from the group consisting of: MIF, DAD1, ARL4, GNS, Transglutaminase 2, Stearyl-CoA-Desaturase and UDP-Glucose Ceramide Glycosyltransferase and thereby affects its biological activity.

In a further aspect, the present invention relates to a method for determining an expression level of a nucleic acid coding for a protein of the invention, preferably messenger RNA, or protein of the invention itself, in a cell, preferably in a macrophage, more preferably in a macrophage isolated from a site of inflammation, even more preferably from a site of inflammation in a subject suffering from a chronic inflammatory airway disease. Such a method can be used, for example, for testing whether a substance is capable of influencing differentially expressed nucleic acid sequence expression levels in a method outlined above for determining whether a substance is an activator or inhibitor according to the present invention. A method for determining an expression level according to the invention can, however, also be used for testing the activation state of a macrophage, e.g. for diagnostic purposes or for investigation of the success of treatment for a disease which is caused by the hyperactivated macrophage. Said macrophage is preferably a mammalian, more preferably a human cell. Accordingly, macrophages of the present invention are preferably obtainable from the site of inflammation in a mammal and more preferably from a site of inflammation in a human being. Accordingly, the invention also relates to a method for diagnosis of a chronic inflammatory disease, or monitoring of such disease, e.g. monitoring success in treating beings in need of treatment for such disease, comprising determining an expression level of a nucleic acid coding for a protein of the invention, preferably messenger RNA, or protein of the invention itself in a macrophage.

A method for determining expression levels of a nucleic acid coding for a protein of the invention, preferably messenger RNA, or protein of the invention itself can, depending on the purpose of determining the expression level, be performed by known procedures such as measuring the concentration of respective RNA transcripts via hybridization techniques or via reporter gene driven assays such as luciferase assays or by measuring the protein concentration of said protein of the invention using respective antibodies.

The present invention also relates to the use of a substance according to the invention for the treatment for a chronic inflammatory airway disease. Another embodiment of the present invention relates to a pharmaceutical composition comprising at least one of the substances according to the invention determined to be an activator or an inhibitor. The composition may be manufactured in a manner that is itself known, e.g. by means of conventional mixing, dissolving, granulating, dragee-making, levigating, powdering, emulsifying, encapsulating, entrapping or lyophilizing processes.

In order to use substances which activate or inhibit according to the invention as drugs for treatment for chronic inflammatory airway diseases, the substances can be tested in animal models, for example, an animal suffering from an inflammatory airway disorder or a transgenic animal expressing protein of the invention.

Toxicity and therapeutic efficacy of a substance according to the invention can be determined by standard pharmaceutical procedures, which include conducting cell culture and animal experiments to determine the IC₅₀, LD₅₀ and ED₅₀. The data obtained are used for estimating the animal or more preferred the human dose range, which will also depend on the dosage form (tablets, capsules, aerosol sprays ampules, etc.) and the administration route (for example transdermal, oral, buccal, nasal, enteral, parenteral, inhalative, intratracheal, or rectal).

A pharmaceutical composition containing at least one substance according to the invention as an active ingredient can be formulated in conventional manner. Methods for making such formulations can be found in manuals, e.g. “Remington Pharmaceutical Science”. Examples for ingredients that are useful for formulating at least one substance according to the present invention are also found in WO 99/18193, which is hereby incorporated by reference.

In a further aspect the invention concerns a method for treating a chronic inflammatory airway disease. Such method comprises administering to a being, preferably to a human being, in need of such treatment a suitable amount of a pharmaceutical composition comprising at least one substance determined to be an activator or inhibitor by a method according to the invention.

In an other embodiment the invention relates to a method for selectively modulating the concentration of a protein of the invention in a macrophage, comprising administering a substance determined to be an activator or inhibitor of protein of the invention.

Included herein are exemplified embodiments, which are intended as illustrations of single aspects of the invention. Indeed, various modifications of the invention in addition to those herein will become apparent to those skilled in the art from the foregoing description. Such modifications are intended to fall within the scope of the present invention.

All publications and patent applications cited herein are incorporated by reference in their entireties.

### EXAMPLES

### Example 1

### Comparative Expression Profiling

The following is an illustration of how comparative expression profiling can be performed in order to identify a protein of the invention.

1.1. Selection of Subjects

Three groups of subjects are studied: healthy non-smokers, healthy smokers and patients with COPD.

In order to assess lung function, subjects have to perform spirometry. A simple calculation based on age and height is used to characterize the results. COPD subjects are included if their FEV₁% (forced expiratory volume, 1 second) predicted is less than 70%. Healthy smokers are age and smoking history matched with the COPD subjects but have normal lung function. Healthy non-smokers have normal lung function and have never smoked. The latter group has a methacholine challenge to exclude asthma. This technique requires increasing doses of methacholine to be given to the subject, with spirometry between each dose. When the FEV₁ falls 20% the test is stopped and the PC₂₀ is calculated. This is the dose of methacholine causing a 20% fall in FEV₁ and we require a value of great er than 32 as evidence of absence of asthma. All subjects have skin prick tests to common allergens and are required to have negative results. This excludes atopic individuals. The clinical history of the subjects is monitored and examined in order to exclude concomitant disease.

1.2. BAL (Bronchoalveolar Lavage) Procedure

Subjects are sedated with midazolam prior to the BAL. Local anaesthetic spray is used to anesthetize the back of the throat. A 7 mm Olympus bronchoscope is used. The lavaged area is the right middle lobe. 250 ml of sterile saline is instilled and immediately aspirated. The resulting aspirate contains macrophages.

1.3. BAL Processing

BAL is filtered through sterile gauze to remove debris. The cells are washed twice in HBSS, resuspended in 1 ml HBSS (Hank's Balanced Salt Solution) and counted. The macrophages are spun to a pellet using 15 ml Falcon blue-cap polypropylene, resuspended in Trizol reagent (Gibco BRL Life Technologies) at a concentration of 1 ml Trizol reagent per 10 million cells and then frozen at −70° C.

1.4. Differential Gene Expression Analysis

Total RNA is extracted from macrophage samples obtained according to Example 1.3. Cell suspensions in Trizol are homogenized through pipetting and incubated at room temperature for 5 minutes. 200 μl chloroform per ml Trizol is added, the mixture carefully mixed for 15 seconds and incubated for 3 more minutes at room temperature. The samples are spun at 10,000 g for 15 minutes at 4° C. The upper phase is transferred into a new reaction tube and the RNA is precipitated by adding 0.5 ml isopropanol per ml Trizol for 10 minutes at room temperature. Then, the precipitate is pelleted by using a microcentrifuge for 10 minutes at 4° C. with 10,000 g, the pellet is washed twice with 75% ethanol, air dried and resuspended in DEPC-H₂O .

An RNA cleanup with Qiagen RNeasy Total RNA isolation kit (Qiagen) is performed in order to improve the purity of the RNA. The purity of the RNA is determined by agarose gel electrophoresis and the concentration is measured by UV absorption at 260 nm.

5 μg of each RNA is used for cDNA synthesis. First and second strand synthesis are performed with the SuperScript Choice system (Gibco BRL Life Technologies). In a total volume of 11 μl RNA and 1 μl of 100 μM T7-(dt)₂₄ primer, sequence shown in SEQ ID NO:15, RNA and primer are heated up to 70° C. for 10 minutes and then cooled down on ice for 2 minutes. First strand buffer to a final concentration of 1×, DTT to a concentration of 10 mM and a dNTP mix to a final concentration of 0.5 mM are added to a total volume of 18 μl. The reaction mix is incubated at 42° C. for 2 minutes and 2 μl of Superscript II reverse transcriptase (200 U/μl) are added. For second strand synthesis 130 μl of a mix containing 1.15× second strand buffer, 230 μM dNTPs, 10 U E. coli DNA ligase (10 U/μl), E. coli DNA polymerase (10 U/μl), RNase H (2 U/μl) is added to the reaction of the first strand synthesis and carefully mixed with a pipette. Second strand synthesis is performed at 16° C. for 2 hours, then 2 μl of T4 DNA polymerase (5 U/μl) are added, incubated for 5 minutes at 16° C. and the reaction is stopped by adding 10 μl 0.5 M EDTA.

Prior to cRNA synthesis the double stranded cDNA is purified. The cDNA is mixed with an equal volume of phenol:chloroform:isoamylalcohol (25:24:1) and spun through the gel matrix of phase lock gels (Eppendorf) in a microcentrifuge in order to separate the cDNA from unbound nucleotides. The aqueous phase is precipitated with ammonium acetate and ethanol. Subsequently, the cDNA is used for in vitro transcription. cRNA synthesis is performed with the ENZO BioArray High Yield RNA Transcript Labeling Kit according to manufacturer's protocol (ENZO Diagnostics). Briefly, the cDNA is incubated with 1× HY reaction buffer, 1× biotin labeled ribonucleotides, 1× DTT, 1× RNase Inhibitor Mix and 1× T7 RNA Polymerase in a total volume of 40 μl for 5 hours at 37° C. Then, the reaction mix is purified via RNeasy columns (Qiagen), the cRNA is precipitated with ammonium acetate and ethanol and finally resuspended in DEPC-treated water. The concentration is determined via UV spectrometry at 260 nm. The remaining cRNA is incubated with 1× fragmentation buffer (5× fragmentation buffer: 200 mM Tris acetate, pH 8.1, 500 mM KOAc, 150 mM MgOAc) at 94° C. for 35 minutes. For hybridization of the DNA chip, 15 μg of cRNA is used, mixed with 50 pM biotin-labeled control B2 oligonucleotide, sequence shown SEQ ID NO:16, 1× cRNA cocktail, 0.1 mg/ml herring sperm DNA, 0.5 mg/ml acetylated BSA, 1× MES (2-[N-morpholino]-ethanesulfonic acid) hybridization buffer in a total volume of 300 μl. The hybridization mixture is heated up to 99° C. for 5 minutes, cooled down to 45° C. for 10 minutes and 200 μl of the mix are used to fill the probe array. The hybridization is performed at 45° C. at 60 rpm for 16 hours.

After the hybridization, the hybridization mix on the chip is replaced by 300 μl non-stringent wash buffer (100 mM MES, 100 mM NaCl, 0.01% Tween 20). The chip is inserted into an Affymetrix Fluidics station and washing and staining is performed according to the EukGE-WS2 protocol. The staining solution per chip consists of 600 μl 1× stain buffer (100 mM MES, 1 M NaCl, 0.05% Tween 20), 2 mg/ml BSA, 10 μg/ml SAPE (streptavidin phycoerythrin) (Dianova), the antibody solution consists of 1× stain buffer, 2 mg/ml BSA, 0.1 mg/ml goat IgG, 3 μg/ml biotinylated antibody.

After the washing and staining procedure the chips are scanned on the HP Gene Array Scanner (Hewlett Packard).

Data Analysis is performed by pair-wise comparisons between chips hybridized with RNA isolated from COPD smokers and chips hybridized with RNA isolated from healthy smokers.

The following is an illustration of differentially expressed genes and their function as identified according to the approach of the present invention.

### Example 2

### MIF

A gene identified as consistently upregulated in individuals with COPD codes for MIF. MIF is secreted by pituitary cells, macrophages, and T cells and its synthesis can be induced by proinflammatory stimuli such as LPS, TNFα, and IFN-γ. MIF itself has proinflammatory activity by counteracting suppressive effects of glucocorticoids and by inducing inflammation in response to invasion of bacteria. Neutralizing MIF can prevent septic shock in certain mouse models (Calandra, T. et al. (1994) J. Exp. Med. 179, 1985-1902; Bernhagen, J. et al. (1998) J. Mol. Med. 76, 151-161; Calandra, T. et al. (2000) Nat. Med. 6, 164-170).

MIF is consistently found upregulated (42%) in COPD smokers compared to healthy smokers. This is shown by fold change “FC” values (Table 1). The p value for comparing COPD smokers and healthy smokers is 0.03.

TABLE 1
Deregulation of MIF: “fold change” (FC) values for each patient are
listed for the comparisons between obstructed and healthy smokers.

| comp | FC | comp | FC | comp | FC | comp | FC |
|---|---|---|---|---|---|---|---|
| 1 vs 2 | −1.3 | 5 vs 43 | 3.9 | 39 vs 57 | −2.0 | 68 vs 66 | 2.8 |
| 1 vs 37 | 8.0 | 5 vs 56 | 1.9 | 39 vs 58 | 1.0 | 68 vs 69 | 2.3 |
| 1 vs 43 | 1.8 | 5 vs 57 | 1.5 | 39 vs 62 | 1.0 | 68 vs 76 | 5.0 |
| 1 vs 56 | −1.3 | 5 vs 58 | 2.9 | 44 vs 2 | 1.4 | 68 vs 78 | 3.2 |
| 1 vs 57 | −1.6 | 5 vs 62 | 2.0 | 44 vs 37 | 14.4 | 70 vs 65 | 1.1 |
| 1 vs 58 | 1.2 | 6 vs 2 | −1.6 | 44 vs 43 | 3.0 | 70 vs 66 | 1.4 |
| 1 vs 62 | −1.2 | 6 vs 37 | 6.5 | 44 vs 56 | 1.4 | 70 vs 69 | 1.1 |
| 3 vs 2 | −1.6 | 6 vs 43 | 1.5 | 44 vs 57 | 1.1 | 70 vs 76 | 2.6 |
| 3 vs 37 | 6.3 | 6 vs 56 | −1.6 | 44 vs 58 | 2.1 | 70 vs 78 | 1.6 |
| 3 vs 43 | 1.4 | 6 vs 57 | −2.0 | 44 vs 62 | 1.5 | 71 vs 65 | 2.1 |
| 3 vs 56 | −1.6 | 6 vs 58 | 1.0 | 64 vs 65 | 2.0 | 71 vs 66 | 2.7 |
| 3 vs 57 | −2.1 | 6 vs 62 | −1.5 | 64 vs 66 | 2.6 | 71 vs 69 | 2.2 |
| 3 vs 58 | −1.1 | 39 vs 2 | −1.6 | 64 vs 69 | 2.1 | 71 vs 76 | 4.9 |
| 3 vs 62 | −1.5 | 39 vs 37 | 1.0 | 64 vs 76 | 4.7 | 71 vs 78 | 3.1 |
| 5 vs 2 | 1.9 | 39 vs 43 | 1.0 | 64 vs 78 | 3.0 | | |
| 5 vs 37 | 18.5 | 39 vs 56 | −1.5 | 68 vs 65 | 2.1 | | |

2.1. Cloning of MIF

MIF is cloned from a total RNA extracted from human THP-1 cells. 5 μg RNA is reverse transcribed into cDNA with 5 ng oligo(dt)₁₈ primer, 1× first strand buffer, 10 mM DTT, 0.5 mM dNTPs and 2 U Superscript II (Gibco BRL) at 42° C. for 50 minutes. Then, the reaction is terminated at 70° C. for 15 minutes and the cDNA concentration is determined by UV-spectrophotometry. For amplification of MIF, 100 ng of the cDNA and 10 pmoles of sequence-specific primers for MIF (forward primer, SEQ ID NO:17 and reverse primer, SEQ ID NO:18) are used for PCR. Reaction conditions are: 2 minutes at 94° C., 35 cycles with 30 seconds at 94° C., 30 seconds at 53° C., 90 seconds at 72° C., followed by 7 minutes at 72° C. with Taq DNA-polymerase. The reaction mix is separated on a 2% agarose gel, a band of about 360 bp is cut out and purified with the QIAEX II extraction kit (Qiagen). The concentration of the purified band is determined and about 120 ng are incubated with 300 ng of pDONR201, the donor vector of the Gateway system (Life Technologies), 1× BP clonase reaction buffer, BP clonase enzyme mix in a total volume of 20 μl for 60 minutes at 25° C. Then, reactions are incubated with 2 μl of proteinase K and incubated for 10 minutes at 37° C. The reaction mix is then electroporated into competent DB3.1 cells and plated on Kanamycin-containing plates. Clones are verified by sequencing. A clone, designated pDONR-MIF, with identical sequence to the database entry (accession no. L19686) is used for further experiments.

2.2. Generation of a Transfection Vector for MIF

The vector containing MIF described under 1.1. is used to transfer the cDNA for MIF to the expression vector pcDNA3.1(+)/attR that contains the “attR1” and “attR2” recombination sites of the Gateway cloning system (Life Technologies) where MIF is expressed under the control of the CMV promoter. 150 ng of the “entry vector” pDONR-MIF is mixed with 150 ng of the “destination vector” pcDNA3.1 (+)/attR, 4 μl of the LR Clonase enzyme mix, 4 μl LR Clonase reaction buffer, added up with TE (Tris/EDTA) to 20 μl and incubated at 25° C. for 60 minutes. Then, 2 μl of proteinase K solution is added and incubated for 10 minutes at 37° C. 1 μl of the reaction mix is transformed into 50 μl DH5α by a heat-shock of 30 seconds at 42° C. after incubating cells with DNA for 30 minutes on ice. After heat-shock of the cells 450 μl of S.O.C. is added and cells are incubated at 37° C. for 60 minutes. Cells (100 μl) are plated on LB plates containing 100 μg/ml ampicillin and incubated overnight.

A colony that contains pcDNA3.1(+)/attR with MIF as an insert is designated pcDNA/MIF and used for transfection studies.

2.3. Expression of Recombinant MIF

The vector containing MIF described under 1.1. is used to transfer the cDNA for MIF to the expression vectors gpET28abc/attR that contains the “attR1” and “attR2” recombination sites of the Gateway cloning system (Life Technologies). These vectors allow the expression of recombinant his-tagged MIF in bacteria under the control of the T7 promoter. 150 ng of the “entry vector” pDONR-MIF is mixed with 150 ng of the “destination vector” gpET28abc/attR, 4 μl of the LR Clonase enzyme mix, 4 μl LR Clonase reaction buffer, added up with TE (Tris/EDTA) to 20 μl and incubated at 25° C. for 60 minutes. Then, 2 μl of proteinase K solution is added and incubated for 10 minutes at 37° C. 1 μl of the reaction mix is transformed into 50 μl DH5α by a heat-shock of 30 seconds at 42° C. after incubating cells with DNA for 30 minutes on ice. After heat-shock of the cells, 450 μl of S.O.C. is added and cells are incubated at 37° C. for 60 minutes. Cells (100 μl) are plated on LB plates containing 100 μg/ml ampicillin and incubated over night.

A colony that contains gpET28abc/attR with MIF fused to the his-tag in the correct reading frame is designated pgPET/MIF and used for expression of MIF in bacteria.

2.4. Purification of Recombinant MIF

One liter LB broth including 100 μg/ml ampicillin is inoculated with 0.5 ml of an overnight culture of E. coli M15(pREP4) that carries pQE/MIF. The culture is incubated at 37° C. with vigorous shaking until OD₆₀₀ of 0.6. Expression is induced by adding 1 mM IPTG and the culture is grown further for 4 hours. Cells are harvested by centrifugation at 4,000× g for 20 minutes at 4° C. The pellet is frozen at −20° C.

Cells are thawed on ice and resuspended in 2 ml/g cell pellet of lysis buffer (50 mM NaH₂PO4, pH 8.0, 300 mM NaCl, 10 mM imidazole). Then, lysozyme is added to 1 mg/ml and incubated on ice for 30 minutes. Then, cells are sonicated (six bursts of 10 seconds at 300 W). 10 μg/ml RNase A and 5 μg/ml DNase I is added and incubated on ice for 10 minutes. Then, lysates are cleared by spinning debris at 10,000× g for 20 minutes at 4° C. Then, protease inhibitors (40 μg/ml bacitracin, 4 μg/ml leupeptin, 4 μg/ml chymostatin, 10 μg/ml pefabloc, 100 μM PMSF) are added. 3 ml of Ni-NTA resin (Qiagen) are added to the lysate and filled into a column. Binding to the resin is allowed for 60 minutes at 4° C. during gentle shaking. Then, column outlet is opened, the resin washed twice with 12 ml wash buffer (50 mM NaH₂PO4, pH 8.0, 300 mM NaCl, 20 mM imidazole) and eluted with four times 3 ml of elution buffer (50 mM NaH₂PO₄, pH 8.0, 300 mM NaCl, 250 mM imidazole). The elution fraction that contains the recombinant protein is determined by SDS-PAGE and protein concentration of the purified protein is determined by the method of Bradford.

2.5. Purification of CD4⁺ T Cells and Mononuclear Cells from Periperal Blood

10 ml blood of healthy volunteers is diluted with 25 ml PBS and layered carefully on top of 15 ml ficoll in a 50 ml Falcon tube. The tube is spun at 400× g for 40 minutes at room temperature. Cells are removed with a pasteur pipet and washed in 50 ml PBS at 500× g for 10 minutes at room temperature (RT).

CD4⁺ lymphocytes are isolated with the help of magnetic beads. The cell fraction (as described in the previous paragraph) is resuspended in 80 μl MACS buffer (PBS, 2 mM EDTA, 0.5% BSA) per 1×10⁷ cells. 20 μl of CD4⁺ separation beads (Miltenyi Biotech) are added to 1×10⁷ cells, mixed and incubated at 4° C. for 15 minutes. Then, 20 volumes of MACS buffer are added and spun at 1,000 rpm for 10 minutes. The pellet is resuspended in 500 μl MACS buffer per 1×10⁸ cells and added to a Miltenyi Separation Column LS⁺ that is equilibrated with 3 ml of MACS buffer. Magnetic beads are exposed to a magnetic field for 30 seconds and labeled CD4⁺ cells are retained. Afterwards, the column is separated from the magnetic field and CD4⁺ cells are flushed out with 5 ml of MACS buffer. Cells are spun down and resuspended in RPMI1640, 10% FCS).

Similarly, human mononuclear cells are isolated from whole blood by ficoll density centrifugation. After seeding, the cells are washed twice in 24 hours with RPMI 1640, 10% FCS in order to remove non-adherent cells.

2.6. Phenotypic/Cellular Effects Caused by MIF

The following assays are performed with cell lines THP-1 (Tsuchiya, S. et al. (1980) Int. J. Cancer 26, 171-176), and MonoMac 6 (Ziegler-Heitbrock, H. W. et al. (1988) Int. J. Cancer 41, 456-461) that are transiently or stably transfected with MIF and the read-outs are compared to mock-transfected cells. In addition, substances according to the invention that stimulate the activity of MIF are added.

Production and Release of Cytokines

Monocytic/macrophage cell lines are stimulated with MIF (1 μg/ml) at cell densities between 2.5 and 5×10⁵ cells/ml. Cells are harvested after 0, 1, 3, 6, 12, 24, 48, and 72 hours, and the supernatant frozen for further investigation. Cells are washed with PBS, and resuspended in 400 μl of RLT buffer (from Qiagen RNeasy Total RNA Isolation Kit) with 143 mM β-mercaptoethanol, the DNA sheared with a 20 g needle for at least 5 times and stored at −70° C.

Stimulation of cells by cigarette smoke is performed using a smoke-enriched media. 100 ml RPMI media without supplements is perfused with the cigarette smoke of 2 cigarettes. The smoke of the cigarettes is pulled into a 50 ml syringe (about 20 volumes of a 50-ml volumes per cigarette) and then perfused into the media. Afterwards, the pH of the media is adjusted to 7.4, and the media is filter sterilized through a 0.2 μm filter. Cells are resuspended in smoke-enriched media and incubated for 10 minutes at 37° C. at a density of 1×10⁶ cells/ml. Then, cells are washed twice with RPMI 1640 and seeded in flasks or 24-well plates (MonoMac6) for the times indicated above.

Total RNAs are isolated with the Qiagen RNeasy Total RNA Isolation Kit (Qiagen) according to the manufacturer's protocol. Purified RNA is used for TaqMan analysis. The expression levels of cytokines TNFα, IL-1β, IL-8, and IL-6 are measured.

Detection of Secreted Cytokines

Proteins in the supernatants of the cultured and stimulated cells are precipitated by adding TCA to a final concentration of 10%. Precipitates are washed twice with 80% ethanol and pellets are resuspended in 50 mM Tris/HCl, pH 7.4, 10 mM MgCl₂, 1 mM EDTA. Protein concentration is determined via the Bradford method and 50 μg of each sample are loaded on 12% SDS polyacrylamide gels. Gels are blotted onto PVDF-membranes, blocked for 1 hour in 5% BSA in TBST, and incubated for 1 hour with commercially available antibodies against human TNFα, IL-1β, IL-8, and IL-6. After washing with TBST blots are incubated with anti-human IgG conjugated to horseradish-peroxidase, washed again and developed with ECL chemiluminescence kit (Amersham). Intensity of the bands are visualized with BioMax X-ray films (Kodak) and quantified by densitometry. Purified CD4⁺ cells (as described in Examples 2.0) are seeded in 96-well-plates (5×10⁴ cells/200 μl) in RPMI 1640, 10% FCS and incubated with dexamethasone (10 nM) in the presence or absence of 10 ng/ml MIF. After 24 hours of incubation at 37° C. in a humidified atmosphere with 5% CO₂, cytokine release (e.g. IL-2 or IFN-γ (interferon-gamma)) is determined by ELISA. MIF overrides the inhibitory effect of dexamethasone and causes release of cytokines. The counteractive effect of MIF on dexamethasone is modulated by adding substances according to the invention (0.1-100 ng/ml) to the reaction mix. The effect is calculated as percent inhibition of the MIF-mediated effect.

In order to determine cytokine release (IL-1β, IL-6, IL-8, TNF-α) in monocytes, the cells need to be treated with 1 μg/ml LPS after 1 hour of preincubation with dexamethasone and MIF (according to previous paragraph).

Detection of Secreted Matrix Metalloproteases and Other Proteases

The procedure is identical to the one used for cytokines. Antibodies used for Western blotting are against human MMP-1, MMP-7, MMP-9, and MMP-12.

Activity of Secreted Matrix Metalloproteases

Protease activity is determined with a fluorescent substrate. Supernatants isolated from stimulated and unstimulated cells (described above) are incubated in a total volume of 50 μl with 1 μM of the substrate (Dabcyl-Gaba-Pro-Gln-Gly-Leu-Glu (EDANS)-Ala-Lys-NH2 (Novabiochem)) for 5 minutes at room temperature. Positive controls are performed with 125 ng purified MMP-12 per reaction. Protease activity is determined by fluorometry with an excitation at 320 nm and an emission at 405 nm.

In an alternative assay to determine proteolytic activity and cell migration, a chemotaxis (Boyden) chamber is used. In the wells of the upper part of the chamber, cells (10⁵ cells per well) are plated on filters coated with an 8 μm layer of Matrigel (Becton Dickinson). In the lower compartment, chemoattractants like MIF (1 μg/ml), leukotriene B₄ (10 ng/ml), MCP-1 (10 ng/ml) are added to the media. After five days, filters are removed, cells on the undersurface that have traversed the Matrigel are fixed with methanol, stained with the Diff-Quik staining kit (Dade Behring) and counted in three high power fields (400×) by light microscopy.

Chemotaxis Assay

In order to determine chemotaxis, a 48 well chemotaxis (Boyden) chamber (Neuroprobe) is used. Cells are starved for 24 hours in RPMI media without FCS. Chemotaxis is stimulated by 100 ng/ml LPS, 10 ng/ml leukotriene B₄,or MCP-1. Addition of MIF (1 μg/ml) is used to block chemotaxis. Substances according to the invention are diluted in RPMI media without FCS and 30 μl is placed in the wells of the lower compartment in order to counteract MIF activity. The upper compartment is separated from the lower compartment by a polycarbonate filter (pore size 8 μm). 50 μl cell suspension (5×10⁴) are placed in the well of the upper compartment. The chamber is incubated for 5 hours at 37° C. in a humidified atmosphere with 5% CO₂. Then, the filter is removed, cells on the upper side are scraped off, cells on the downside are fixed for 5 minutes in methanol and stained with the Diff-Quik staining set (Dade Behring). Migrated cells are counted in three high-power fields (400×) by light microscopy.

Adherence Assay

Cells are harvested, washed in PBS and resuspended (4×10⁶/ml) in PBS and 1 μM BCECF ((2′-7′-bis-(carboxyethyl)-5(6′)-carboxyfluorescein acetoxymethyl) ester, Calbiochem) and incubated for 20 minutes at 37° C. Cells are washed in PBS and resuspended (3.3×10⁶/ml) in PBS containing 0.1% BSA. 3×10⁵ cells (90 μl) are added to each well of a 96-well flat bottom plate coated with laminin (Becton Dickinson) and allowed to settle for 10 minutes. Substances according to the invention are added in the presence and absence of MIF (1 μg/ml), and plates are incubated for 20 minutes at 37° C. Cells are washed with PBS containing 0.1% BSA and adherent cells are solubilized with 100 μl of 0.025 M NaOH and 0.1% SDS. Quantification is performed by fluorescence measurement.

Phagocytosis

Cell suspensions (2.5×10⁴ cells/ml) are seeded in 6-well plates with 5 ml of U937 or THP-1 in 24-well plates with 2 ml of MonoMac6 and incubated for 1 hour at 37° C. in a humidified atmosphere with 5% CO₂. In the presence of MIF, substances according to the invention are added to counteract the activity of MIF. 40 μl of a dispersed suspension of heat-inactivated Saccharomyces boulardii (20 yeast/cell) are added to each well. Cells are incubated for three more hours, washed twice with PBS and cytocentrifuged. The cytospin preparations are stained with May-Grünwald-Giemsa and phagocytosed particles are counted by light microscopy.

### Example 3

### DAD1

A gene identified as being downregulated in COPD smokers compared to healthy smokers is DAD1 (defender against apoptotic cell death 1). Originally, DAD1 was discovered as being a negative regulator of apoptosis (Nakashima et al. 1993). By homology to the Ost2 protein in Schizosaccaromyces pombe it was identified as the 16 kDa subunit of the oligosaccaryltransferase complex which catalyzes the transfer of high mannose oligosaccharides onto asparagine residues in nascent polypeptides. DAD1 is an integral membrane protein and is ubiquitously expressed (Kelleher, D. J. and R. Gilmore (1997) Proc. Natl. Acad. Sci. USA 94(10):4994-4999).

DAD1 is consistently found upregulated (42%) in comparisons between COPD smokers and healthy smokers. This is shown by “fold change” values (Table 2).

TABLE 2
Fold change values (FC) for comparisons between obstructed
smokers and healthy smokers. On average DAD1 is upregulated
by 1.6 fold, the median is 1.5 fold.

| comp | FC | comp | FC | comp | FC | comp | FC |
|---|---|---|---|---|---|---|---|
| 1 vs 2 | −1.1 | 5 vs 43 | 2.3 | 39 vs 57 | 4.8 | 68 vs 66 | 1.4 |
| 1 vs 37 | 2.5 | 5 vs 56 | 3.9 | 39 vs 58 | 2.5 | 68 vs 69 | 1. |
| 1 vs 43 | 1.5 | 5 vs 57 | 4.0 | 39 vs 62 | 6.6 | 68 vs 76 | 2.2 |
| 1 vs 56 | 2.4 | 5 vs 58 | 2.0 | 44 vs 2 | −2.9 | 68 vs 78 | 2.1 |
| 1 vs 57 | 2.5 | 5 vs 62 | 5.5 | 44 vs 37 | 1.1 | 70 vs 65 | −1.3 |
| 1 vs 58 | 1.3 | 6 vs 2 | 1.0 | 44 vs 43 | −1.7 | 70 vs 66 | −1.4 |
| 1 vs 62 | 3.4 | 6 vs 37 | 2.7 | 44 vs 56 | 1.0 | 70 vs 69 | −1.3 |
| 3 vs 2 | −1.2 | 6 vs 43 | 1.6 | 44 vs 57 | 1.0 | 70 vs 76 | 1.1 |
| 3 vs 37 | 2.3 | 6 vs 56 | 2.7 | 44 vs 58 | −1.9 | 70 vs 78 | 1.1 |
| 3 vs 43 | 1.4 | 6 vs 57 | 2.7 | 44 vs 62 | 1.4 | 71 vs 65 | 1.1 |
| 3 vs 56 | 2.3 | 6 vs 58 | 1.4 | 64 vs 65 | −1.1 | 71 vs 66 | 1.0 |
| 3 vs 57 | 2.3 | 6 vs 62 | 3.7 | 64 vs 66 | −1.1 | 71 vs 69 | 1.2 |
| 3 vs 58 | 1.2 | 39 vs 2 | 1.7 | 64 vs 69 | −1.1 | 71 vs 76 | 1.6 |
| 3 vs 62 | 3.2 | 39 vs 37 | 4.8 | 64 vs 76 | 1.3 | 71 vs 78 | 1.6 |
| 5 vs 2 | 1.4 | 39 vs 43 | 2.8 | 64 vs 78 | 1.3 | | |
| 5 vs 37 | 3.9 | 39 vs 56 | 4.7 | 68 vs 65 | 1.4 | | |

The protein is cloned and assays are designed and performed in an analogous manner to the cloning and assays described hereinbefore.

### Example 4

### ARL4

A gene identified as being upregulated in COPD smokers compared to healthy smokers is ARL4 (ADP-ribosylation factor-like protein 4). ARLs belong to the family of ADP-ribosylation factors (ARFs). ARFs are involved in vesicular and membrane trafficking. ARL4 is both detected inside and outside of the nucleus and it is speculated that it is involved in cellular differentiation (Jacobs, S. et al. (1999) FEBS Lett. 456(3):384-388).

ARL4 is consistently found upregulated (45%) in comparisons between COPD smokers and healthy smokers. This is shown by “fold change” values (Table 3). The p values for two separate groups comparing COPD smokers and healthy smokers are 0.10 and 0.06.

TABLE 3
Fold change values (FC) for comparisons between obstructed
smokers and healthy smokers. On average ARL4 is upregulated
by 1.6 fold, the median is 1.9 fold.

| comp | FC | comp | FC | comp | FC | comp | FC |
|---|---|---|---|---|---|---|---|
| 1 vs 2 | −1.1 | 5 vs 43 | 1.9 | 39 vs 57 | 2.5 | 68 vs 66 | 2.4 |
| 1 vs 37 | 2.7 | 5 vs 56 | 2.2 | 39 vs 58 | 1.2 | 68 vs 69 | 4.5 |
| 1 vs 43 | 3.2 | 5 vs 57 | 1.6 | 39 vs 62 | 1.5 | 68 vs 76 | 7.8 |
| 1 vs 56 | 4.3 | 5 vs 58 | −1.2 | 44 vs 2 | −3.7 | 68 vs 78 | 3.3 |
| 1 vs 57 | 2.0 | 5 vs 62 | 1.0 | 44 vs 37 | −1.3 | 70 vs 65 | 1.2 |
| 1 vs 58 | −1.1 | 6 vs 2 | 1.2 | 44 vs 43 | −1.1 | 70 vs 66 | 1.5 |
| 1 vs 62 | 1.2 | 6 vs 37 | 3.4 | 44 vs 56 | 1.5 | 70 vs 69 | 2.7 |
| 3 vs 2 | −1.8 | 6 vs 43 | 3.6 | 44 vs 57 | −1.7 | 70 vs 76 | 4.7 |
| 3 vs 37 | 2.0 | 6 vs 56 | 4.1 | 44 vs 58 | −3.5 | 70 vs 78 | 1.9 |
| 3 vs 43 | 2.4 | 6 vs 57 | 2.7 | 44 vs 62 | −2.7 | 71 vs 65 | 1.7 |
| 3 vs 56 | 3.2 | 6 vs 58 | 1.3 | 64 vs 65 | −1.1 | 71 vs 66 | 2.0 |
| 3 vs 57 | 1.5 | 6 vs 62 | 1.6 | 64 vs 66 | 1.2 | 71 vs 69 | 3.9 |
| 3 vs 58 | −1.4 | 39 vs 2 | 1.1 | 64 vs 69 | 2.2 | 71 vs 76 | 6.7 |
| 3 vs 62 | 1.0 | 39 vs 37 | 3.3 | 64 vs 76 | 3.8 | 71 vs 78 | 2.8 |
| 5 vs 2 | −1.3 | 39 vs 43 | 4.0 | 64 vs 78 | 1.6 | | |
| 5 vs 37 | 1.8 | 39 vs 56 | 4.7 | 68 vs 65 | 1.9 | | |

4.1. Cloning of ARL4

ARL4 is cloned from total RNA extracted from human 3T3-L1. 5 μg RNA is reverse transcribed into cDNA with 5 ng oligo(dt)₁₈ primer, 1× first strand buffer, 10 mM DTT, 0.5 mM dNTPs and 2 U Superscript II (Gibco BRL) at 42° C. for 50 minutes. Then, the reaction is terminated at 70° C. for 15 minutes and the cDNA concentration is determined by UV-spectrophotometry. For amplification of ARL4, 100 ng of the cDNA and 10 pmoles of sequence-specific primers for ARL4 (forward primer, SEQ ID NO:19 and reverse primer, SEQ ID NO:20) are used for PCR. Reaction conditions are: 2 minutes at 94° C., 35 cycles with 30 seconds at 94° C., 30 seconds at 53° C., 90 seconds at 72° C., followed by 7 minutes at 72° C. with Taq DNA-polymerase. The PCR product is separated on a 2% agarose gel, a band of about 600 bp is cut out and purified with the QIAEX II extraction kit (Qiagen). This product is digested with BamH1 and HindIII and cloned into pQE-30 (Qiagen) that is digested with BamHI and HindIII. A clone, designated pQE/ARL4 with identical sequence to the database entry (acc. U73960) is used for further experiments.

4.2 Expression of ARL4

One liter LB broth including 100 μg/ml ampicillin is inoculated with 0.5 ml of an overnight culture of E. coli M15(pREP4) that carries pQE/ARL4. The culture is incubated at 37° C. with vigorous shaking until OD₆₀₀ of 0.6. Expression is induced by adding 1 mM IPTG and the culture is grown further for 4 hours. Cells are harvested by centrifugation at 4,000× g for 20 minutes at 4° C. The pellet is frozen at −20° C.

Cells are thawed on ice and resuspended in 2 ml/g cell pellet of lysis buffer (50 mM NaH₂PO4, pH 8.0, 300 mM NaCl, 10 mM imidazole). Then, lysozyme is added to 1 mg/ml and incubated on ice for 30 minutes. Then, cells are sonicated (six bursts of 10 seconds at 300 W). 10 μg/ml RNase A and 5 μg/ml DNase I is added and incubated on ice for 10 minutes. Then, lysates are cleared by spinning debris at 10,000× g for 20 minutes at 4° C. Then, protease inhibitors (40 μg/ml bacitracin, 4 μg/ml leupeptin, 4 μg/ml chymostatin, 10 μg/ml pefabloc, 100 μM PMSF) are added. 3 ml of Ni-NTA resin (Qiagen) are added to the lysate and filled into a column. Binding to the resin is allowed for 60 minutes at 4° C. during gentle shaking. Then, the column outlet is opened, the resin washed twice with 12 ml wash buffer (50 mM NaH₂PO4, pH 8.0, 300 mM NaCl, 20 mM imidazole) and eluted with four times 3 ml of elution buffer (50 mM NaH₂PO4, pH 8.0, 300 mM NaCl, 250 mM imidazole). The elution fraction that contains the recombinant protein is determined by SDS-PAGE and protein concentration of the purified protein is determined by the method of Bradford.

4.3 GTPγS Binding Assay

Recombinant ARL4 (1 μM) is incubated at 37° C. with [³⁵S]GTPS or [³H]GDP (10 μM, approximately 1,000 cpm/pmol) in 50 mM Hepes (pH7.5), 1 mM dithiothreitol, 1 mM MgCl2 with or without (as indicated in the figure legends) 2 mM EDTA (1 μM or 1 mM free Mg⁺⁺), 100 mM KCl. Substances according to the invention are preincubated with ARL4 for 5 minutes at 4° C. in a concentration range from 0.5 to 300 nM before starting the GTPγS binding reaction. At various time points (10 seconds to 30 minutes) samples of 25 μl (25 pmoles of ARF) are removed, diluted into 2 ml of ice-cold 20 mM Hepes (pH 7.5), 100 mM NaCl, and 10 mM MgCl₂, and filtered on 25-mm BA 85 nitrocellulose filters (Schleicher & Schüll). Filters are washed twice with 2 ml of the same buffer, dried, and quantified by scintillation counting.

### Example 5

### GNS.

A gene identified as being downregulated in COPD smokers compared to healthy smokers is Glucosamine-6-sulphatase (GNS). GNS hydrolyzes the 6-sulfate group of the N-acetyl-d-glucosamine 6-sulfate units of heparan (Kresse, H. et al. (1980) Proc. Natl. Acad. Sci. U.S.A. 77, 6822-6826). GNS is consistently found downregulated (44%) in comparisons between COPD smokers and healthy smokers. This is shown by “fold change” values (Table 4). The p values for two separate groups comparing COPD smokers and healthy smokers are 0.05 and 0.006.

TABLE 4
Fold change values (FC) for comparisons between obstructed
smokers and healthy smokers. On average GNS is downregulated
by −2.0 fold, the median is −1.8 fold

| comp | FC | comp | FC | comp | FC | comp | FC |
|---|---|---|---|---|---|---|---|
| 1 vs 2 | 1.0 | 5 vs 43 | −4.6 | 39 vs 57 | −2.4 | 68 vs 66 | −3.6 |
| 1 vs 37 | 1.0 | 5 vs 56 | −1.7 | 39 vs 58 | −3.3 | 68 vs 69 | −2.3 |
| 1 vs 43 | −3.7 | 5 vs 57 | −3.1 | 39 vs 62 | −1.1 | 68 vs 76 | −2.6 |
| 1 vs 56 | −1.1 | 5 vs 58 | −4.0 | 44 vs 2 | −1.2 | 68 vs 78 | −2.6 |
| 1 vs 57 | −2.3 | 5 vs 62 | 1.0 | 44 vs 37 | −1.2 | 70 vs 65 | −1.4 |
| 1 vs 58 | −3.0 | 6 vs 2 | 1.0 | 44 vs 43 | −4.3 | 70 vs 66 | −1.6 |
| 1 vs 62 | 1.0 | 6 vs 37 | 1.1 | 44 vs 56 | −1.3 | 70 vs 69 | 1.0 |
| 3 vs 2 | −1.5 | 6 vs 43 | −3.5 | 44 vs 57 | −2.6 | 70 vs 76 | −1.1 |
| 3 vs 37 | −1.4 | 6 vs 56 | 1.0 | 44 vs 58 | −3.7 | 70 vs 78 | −1.1 |
| 3 vs 43 | −5.0 | 6 vs 57 | −2.2 | 44 vs 62 | −1.2 | 71 vs 65 | −2.1 |
| 3 vs 56 | −1.8 | 6 vs 58 | −3.0 | 64 vs 65 | −2.3 | 71 vs 66 | −2.5 |
| 3 vs 57 | −3.1 | 6 vs 62 | 1.1 | 64 vs 66 | −2.6 | 71 vs 69 | −1.7 |
| 3 vs 58 | −3.9 | 39 vs 2 | 1.0 | 64 vs 69 | −1.7 | 71 vs 76 | −1.8 |
| 3 vs 62 | −1.3 | 39 vs 37 | −1.1 | 64 vs 76 | −1.9 | 71 vs 78 | −1.8 |
| 5 vs 2 | −1.7 | 39 vs 43 | −3.8 | 64 vs 78 | −1.9 | | |
| 5 vs 37 | −1.7 | 39 vs 56 | 1.0 | 68 vs 65 | −3.1 | | |

The protein is cloned and assays are designed and performed in an analogous manner to the cloning and assays described hereinbefore.

### Example 6

### Transglutaminase 2

A gene identified as being downregulated in COPD smokers compared to healthy smokers is transglutaminase 2. This enzyme belongs to a family of calcium-dependent transglutaminases that catalyze the covalent cross-linking of specific proteins by the formulation of (γ-glutamyl)lysine bonds and the conjugation of polyamines to proteins (Folk, J. E. (1980) Annu. Rev. Biochem. 49, 517-531). Transglutaminases can also be secreted. The physiological functions are not well understood, it may be involved in the specialized processing of the matrix that occurs during bone formation, wound healing, and other remodeling processes (Lu, S. et al. (1995) J. Biol. Chem. 270, 9748-9756).

Transglutaminase 2 is consistently found downregulated (55%) in comparisons between COPD smokers and healthy smokers. This is shown by “fold change” values (Table 5). The p values for two separate groups comparing COPD smokers and healthy smokers are 0.04 and 0.16.

TABLE 5
Fold change values (FC) for comparisons between obstructed
smoker and healthy smokers. On average Transglutaminase 2 is
downregulated by 2.3 fold,the median is −2.35 fold

| comp | FC | comp | FC | comp | FC | comp | FC |
|---|---|---|---|---|---|---|---|
| 1 vs 2 | 1.0 | 5 vs 43 | −5.6 | 39 vs 57 | −2.3 | 68 vs 66 | −2.8 |
| 1 vs 37 | −3.6 | 5 vs 56 | −1.4 | 39 vs 58 | −3.9 | 68 vs 69 | −7.4 |
| 1 vs 43 | −6.9 | 5 vs 57 | −3.7 | 39 vs 62 | 1.0 | 68 vs 76 | −4−4 |
| 1 vs 56 | −1.5 | 5 vs 58 | −7.5 | 44 vs 2 | 1.0 | 68 vs 78 | −3.4 |
| 1 vs 57 | −3.6 | 5 vs 62 | 1.0 | 44 vs 37 | −3.2 | 70 vs 65 | 1.5 |
| 1 vs 58 | −8.9 | 6 vs 2 | 2.2 | 44 vs 43 | −7.7 | 70 vs 66 | 1.2 |
| 1 vs 62 | 1.0 | 6 vs 37 | −2.2 | 44 vs 56 | −1.9 | 70 vs 69 | −2.5 |
| 3 vs 2 | 1.0 | 6 vs 43 | −3.6 | 44 vs 57 | −3.8 | 70 vs 76 | −1.4 |
| 3 vs 37 | −2.5 | 6 vs 56 | 1.0 | 44 vs 58 | −11.3 | 70 vs 78 | 1.0 |
| 3 vs 43 | −4.5 | 6 vs 57 | −2.5 | 44 vs 62 | 1.0 | 71 vs 65 | −1.8 |
| 3 vs 56 | −1.2 | 6 vs 58 | −4.7 | 64 vs 65 | 1.4 | 71 vs 66 | −2.4 |
| 3 vs 57 | −2.8 | 6 vs 62 | −1.2 | 64 vs 66 | 1.1 | 71 vs 69 | −6.9 |
| 3 vs 58 | −5.9 | 39 vs 2 | 1.0 | 64 vs 69 | −2.7 | 71 vs 76 | −3.9 |
| 3 vs 62 | 1.0 | 39 vs 37 | −1.8 | 64 vs 76 | −1.5 | 71 vs 78 | −2.8 |
| 5 vs 2 | 1.0 | 39 vs 43 | −2.9 | 64 vs 78 | −1.1 | | |
| 5 vs 37 | −3.3 | 39 vs 56 | 1.2 | 68 vs 65 | −2.1 | | |

The protein is cloned and assays are designed and performed in an analogous manner to the cloning and assays described hereinbefore.

### Example 7

### Stearyl-CoA-Desaturase

A gene identified as being downregulated in COPD smokers compared to healthy smokers is Stearoyl-CoA-Desaturase. Stearoyl-CoA-Desaturase catalyzes the oxidation of palmitoyl-CoA and stearoyl-CoA at the Δ⁹ position to form the mono-unsaturated fatty acyl-CoA esters, palmitoleoyl-CoA and aoleoyl-CoA, respectively (Enoch, H. G. et al. (1976) J. Biol. Chem. 251, 5095-5103).

Stearoyl-CoA-desaturase is consistently found downregulated (48%) in comparisons between COPD smokers and healthy smokers. This is shown by “fold change” values (Table 6). The p values for two separate groups comparing COPD smokers and healthy smokers are 0.03 and 0.15.

TABLE 6
Fold change values (FC) for comparisons between obstructed
smokers and healthy smokers. On average Stearoyl-CoA-desaturase
is downregulated by 2.3 fold, the median is −1.9 fold

| comp | FC | comp | FC | comp | FC | comp | FC |
|---|---|---|---|---|---|---|---|
| 1 vs 2 | −1.7 | 5 vs 43 | −5.8 | 39 vs 57 | −3.9 | 68 vs 66 | −2.5 |
| 1 vs 37 | 1.0 | 5 vs 56 | −2.1 | 39 vs 58 | −7.3 | 68 vs 69 | −1.2 |
| 1 vs 43 | −4.0 | 5 vs 57 | −3.7 | 39 vs 62 | −1.8 | 68 vs 76 | −1.2 |
| 1 vs 56 | 1.0 | 5 vs 58 | −6.5 | 44 vs 2 | −1.1 | 68 vs 78 | −1.5 |
| 1 vs 57 | −2.4 | 5 vs 62 | −2.3 | 44 vs 37 | 1.3 | 70 vs 65 | −1.5 |
| 1 vs 58 | −4.6 | 6 vs 2 | −3.0 | 44 vs 43 | −2.4 | 70 vs 66 | −1.2 |
| 1 vs 62 | −1.1 | 6 vs 37 | −1.8 | 44 vs 56 | 1.4 | 70 vs 69 | 1.5 |
| 3 vs 2 | −1.8 | 6 vs 43 | −7.1 | 44 vs 57 | −1.5 | 70 vs 76 | 1.5 |
| 3 vs 37 | −1.1 | 6 vs 56 | −2.2 | 44 vs 58 | −2.9 | 70 vs 78 | 1.3 |
| 3 vs 43 | −4.4 | 6 vs 57 | −4.3 | 44 vs 62 | 1.3 | 71 vs 65 | −2.5 |
| 3 vs 56 | −1.2 | 6 vs 58 | −8.2 | 64 vs 65 | −4.2 | 71 vs 66 | −1.9 |
| 3 vs 57 | −2.7 | 6 vs 62 | −2.4 | 64 vs 66 | −3.3 | 71 vs 69 | 1.0 |
| 3 vs 58 | −5.0 | 39 vs 2 | −2.7 | 64 vs 69 | −1.7 | 71 vs 76 | −1.1 |
| 3 vs 62 | −1.2 | 39 vs 37 | −1.6 | 64 vs 76 | −1.7 | 71 vs 78 | −1.3 |
| 5 vs 2 | −2.9 | 39 vs 43 | −6.4 | 64 vs 78 | −2.2 | | |
| 5 vs 37 | −1.9 | 39 vs 56 | −1.7 | 68 vs 65 | −3.3 | | |

The protein is cloned and assays are designed and performed in an analogous manner to the cloning and assays described hereinbefore.

### Example 8

### UDP-Glucose Ceramide Glycosyltransferase

A gene identified as being downregulated in COPD smokers compared to healthy smokers is UDP-glucose Ceramide Glucosyltransferase. This enzyme catalyzes the transfer of glucose from UDP-glucose to ceramide. The product glucosyl-Stearoyl-CoA-desaturase ceramid serves as the core structure of more than 300 glycosphingolipids that are involved in multiple cellular processes as differentiation, adhesion, proliferation, and cell-cell recognition (Basu, S. et al. (1968) J. Biol. Chem. 243, 5802-5807; Ichikawa, S. et al. (1996) Proc. Natl. Acad. Sci. U.S.A. 93, 4638-4643).

Ceramide Glucosyltransferase is consistently found downregulated (48%) in comparisons between COPD smokers and healthy smokers. This is shown by “fold change” values (Table 7).

TABLE 7
Fold change values (FC) for comparisons between obstructed
smokers and healthy smokers. On average Ceramide Glucosyltransferase
is downregulated by 1.2 fold, the median is −1.9 fold

| comp | FC | comp | FC | comp | FC | comp | FC |
|---|---|---|---|---|---|---|---|
| 1 vs 2 | 1.3 | 5 vs 43 | −2.4 | 39 vs 57 | −1.6 | 68 vs 66 | −4.0 |
| 1 vs 37 | −2.4 | 5 vs 56 | −2.0 | 39 vs 58 | −2.6 | 68 vs 69 | −1.1 |
| 1 vs 43 | −1.9 | 5 vs 57 | −1.6 | 39 vs 62 | −2.3 | 68 vs 76 | −2.9 |
| 1 vs 56 | −1.5 | 5 vs 58 | −2.6 | 44 vs 2 | 7.2 | 68 vs 78 | −3.4 |
| 1 vs 57 | −1.3 | 5 vs 62 | −2.0 | 44 vs 37 | 1.9 | 70 vs 65 | 1.0 |
| 1 vs 58 | −2.1 | 6 vs 2 | 1.0 | 44 vs 43 | 2.7 | 70 vs 66 | −2.0 |
| 1 vs 62 | −1.5 | 6 vs 37 | −4.2 | 44 vs 56 | 3.5 | 70 vs 69 | 1.5 |
| 3 vs 2 | 1.3 | 6 vs 43 | −2.8 | 44 vs 57 | 4.6 | 70 vs 76 | −1.4 |
| 3 vs 37 | −2.6 | 6 vs 56 | −2.3 | 44 vs 58 | 2.7 | 70 vs 78 | −1.8 |
| 3 vs 43 | −1.9 | 6 vs 57 | −1.8 | 44 vs 62 | 3.4 | 71 vs 65 | −2.0 |
| 3 vs 56 | −1.6 | 6 vs 58 | −3.0 | 64 vs 65 | −1.7 | 71 vs 66 | −4.3 |
| 3 vs 57 | −1.3 | 6 vs 62 | −2.4 | 64 vs 66 | −3.2 | 71 vs 69 | 1.0 |
| 3 vs 58 | −2.1 | 39 vs 2 | 1.0 | 64 vs 69 | −1.1 | 71 vs 76 | −2.5 |
| 3 vs 62 | −1.7 | 39 vs 37 | −3.5 | 64 vs 76 | −2.5 | 71 vs 78 | −3.7 |
| 5 vs 2 | 1.0 | 39 vs 43 | −2.4 | 64 vs 78 | −2.9 | | |
| 5 vs 37 | −3.1 | 39 vs 56 | −2.2 | 68 vs 65 | −1.9 | | |

The protein is cloned and assays are designed and performed in an analogous manner to the cloning and assays described hereinbefore.

What is claimed is:

1. A method for determining whether a substance is an activator or an inhibitor of a function of a protein comprising: (a) contacting the protein with a substance to be tested, wherein the protein is selected from the group consisting of: MIF, DAD1, ARL4, GNS, Transglutaminase 2, Stearyl-CoA-Desaturase and UDP-Glucose Ceramide Glycosyltransferase; and (b) measuring whether the function is inhibited or activated.

2. A method for determining whether a substance is an activator or an inhibitor of a function of a protein comprising: (a) contacting the protein with a substance to be tested, wherein the protein is a functionally equivalent variant, mutant or fragment of a protein selected from the group consisting of: MIF, DAD1, ARL4, GNS, Transglutaminase 2, Stearyl-CoA-Desaturase and UDP-Glucose Ceramide Glycosyltransferase; and (b) measuring whether the function is inhibited or activated.

3. The method according to claim 1 wherein the inhibition or activation of the function is measured directly.

4. The method according to claim 1 wherein the inhibition or activation of the function is measured indirectly.

5. The method according to claim 1 wherein the protein is a mammalian protein.

6. The method according to claim 5 wherein the protein is a human protein.

7. The method according to claim 1 wherein the method is performed using a cellular system.

8. The method according to claim 1 wherein the method is performed using a cell-free system.

9. A method for determining an expression level of a protein comprising: (a) determining the level of the protein in a hyperactivated macrophage, wherein the protein is selected from the group consisting of: MIF, DAD1, ARL4, GNS, Transglutaminase 2, Stearyl-CoA-Desaturase and UDP-Glucose Ceramide Glycosyltransferase; (b) determining the level of the protein in a non-hyperactivated macrophage, wherein the protein is selected from the group consisting of: MIF, DAD1, ARL4, GNS, Transglutaminase 2, Stearyl-CoA-Desaturase and UDP-Glucose Ceramide Glycosyltransferase; and (c) comparing the level of the protein expressed in step (a) to the level of the protein expressed in step (b), wherein a difference in levels indicates a differentially expressed protein.

10. The method according to claim 9 wherein the hyperactived macrophage is a mammalian macrophage and the non-hyperactivated macrophage is a mammalian macrophage.

11. The method according to claim 10 wherein the hyperactived macrophage is a human macrophage and the non-hyperactivated macrophage is a human macrophage.

12. A method for diagnosing or monitoring a chronic inflammatory airway disease comprising: (a) determining the level of the protein in a hyperactivated macrophage, wherein the protein is selected from the group consisting of: MIF, DAD1, ARL4, GNS, Transglutaminase 2, Stearyl-CoA-Desaturase and UDP-Glucose Ceramide Glycosyltransferase; (b) determining the level of the protein in a non-hyperactivated macrophage, wherein the protein is selected from the group consisting of: MIF, DAD1, ARL4, GNS, Transglutaminase 2, Stearyl-CoA-Desaturase and UDP-Glucose Ceramide Glycosyltransferase; and (c) comparing the level of the protein expressed in step (a) to the level of the protein expressed in step (b), wherein a difference in levels indicates a differentially expressed protein.

13. The method according to claim 12 wherein the chronic inflammatory airway disease is selected from the group consisting of: chronic bronchitis and COPD

14. A substance determined to be an activator or inhibitor of a protein selected from the group consisting of: MIF, DAD1, ARL4, GNS, Transglutaminase 2, Stearyl-CoA-Desaturase and UDP-Glucose Ceramide Glycosyltransferase.

15. A substance determined to be an activator or an inhibitor of a protein selected from the group consisting of: MIF, DAD1, ARL4, GNS, Transglutaminase 2, Stearyl-CoA-Desaturase and UDP-Glucose Ceramide Glycosyltransferase according to the method of claim 1.

16. A substance for the treatment of a disease wherein the substance is an activator or an inhibitor of a protein selected from the group consisting of: MIF, DAD1, ARL4, GNS, Transglutaminase 2, Stearyl-CoA-Desaturase and UDP-Glucose Ceramide Glycosyltransferase

17. The substance according to claim 16 wherein the disease is a chronic inflammatory airway disease.

18. The substance according to claim 17 wherein the chronic inflammatory airway disease is selected from the group consisting of: chronic bronchitis and COPD.

19. A pharmaceutical composition comprising at least one substance determined to be an activator or an inhibitor of a protein selected from the group consisting of MIF, DAD1, ARL4, GNS, Transglutaminase 2, Stearyl-CoA-Desaturase and UDP-Glucose Ceramide Glycosyltransferase.

20. A pharmaceutical composition comprising at least one substance determined to be an activator or an inhibitor of a protein selected from the group consisting of MIF, DAD1, ARL4, GNS, Transglutaminase 2, Stearyl-CoA-Desaturase and UDP-Glucose Ceramide Glycosyltransferase according to the method of claim 1.

21. A method for treating a chronic inflammatory airway disease comprising: administering to a subject in need of such treatment an effective amount of a pharmaceutical composition comprising at least one substance determined to be an activator or an inhibitor of a protein selected from the group consisting of: MIF, DAD1, ARL4, GNS, Transglutaminase 2, Stearyl-CoA-Desaturase and UDP-Glucose Ceramide Glycosyltransferase.

22. A method for treating a chronic inflammatory airway disease comprising: administering to a subject in need of such treatment an effective amount of a pharmaceutical composition comprising at least one substance determined to be an activator or an inhibitor of a protein selected from the group consisting of: MIF, DAD1, ARL4, GNS, Transglutaminase 2, Stearyl-CoA-Desaturase and UDP-Glucose Ceramide Glycosyltransferase according to the method of claim 1.

23. The method according to claim 21 wherein the subject is a mammal.

24. The method according to claim 21 wherein the subject is a human.

25. The method according to claim 21 wherein the chronic inflammatory airway disease is selected from the group consisting of: chronic bronchitis and COPD.

26. A method for selectively modulating a protein selected from the group consisting of MIF, DAD1, ARL4, GNS, Transglutaminase 2, Stearyl-CoA-Desaturase and UDP-Glucose Ceramide Glycosyltransferase in a macrophage, comprising administering a substance determined to be an activator or an inhibitor of a protein selected from the group consisting of MIF, DAD1, ARL4, GNS, Transglutaminase 2, Stearyl-CoA-Desaturase and UDP-Glucose Ceramide Glycosyltransferase.

27. The method according to claim 26 wherein the macrophage is involved in a chronic inflammatory airway disease.

28. The method according to claim 27 wherein the chronic inflammatory airway disease is selected from the group consisting of: chronic bronchitis and COPD.

29. A method for selectively modulating a protein selected from the group consisting of MIF, DAD1, ARL4, GNS, Transglutaminase 2, Stearyl-CoA-Desaturase and UDP-Glucose Ceramide Glycosyltransferase in a macrophage, comprising administering a substance determined to be an activator or an inhibitor of a protein selected from the group consisting of MIF, DAD1, ARL4, GNS, Transglutaminase 2, Stearyl-CoA-Desaturase and UDP-Glucose Ceramide Glycosyltransferase according to the method of claim 1.


SEQUENCE LISTING (20 sequences)

SEQ ID NO: 1 - DNA, 2167 bp, Homo sapiens
ctgcaggaaccaatacccataggctatttgtataaatgggccatggggcctcccagctggaggctggctggtgccacgagggtcccacaggcatgggtgtccttcctatatcacatggccttcactgagactggtatatggattgcacctatcagagaccaaggacaggacctccctggaaatctctgaggacctggcctgtgatccagttgctgccttgtcctcttcctgctatgtcatggcttatcttctttcacccattcattcattcattcattcattcagcagtattagtcaatgtctcttgatatgcctggcacctgctagatggtccccgagtttaccattagtggaaaagacatttaagaaattcaccaagggctctatgagaggccatacacggtggacctgactagggtgtggcttccctgaggagctgaagttgcccagaggcccagagaaggggagctgagcacgtttgaaccactgaacctgctctggacctcgcctccttccttcggtgcctcccagcatcctatcctctttaaagagcaggggttcagggaagttccctggatggtgattcgcaggggcagctcccctctcacctgccgcatgactaccccgccccatctcaaacacacaagctcacgcatgcgggactggagcccttgaggacatgtggcccaaagacaggaggtacaggggctcagtgcgtgcagtggaatgaactgggcttcatctctggaagggtaaggggccatcttccgggttcaccgccgcatccccacccccggcacagcgcctcctggcgactaacatcggtgacttagtgaaaggactaagaaagacccgaggcgaggccggaacaggccgatttctagccgccaagtggagaacaggttggagcggtgcgccgggcttagcggcggttgctggaggaacgggcggagtcgcccagggtcctgccctgcgggggtcgagccgaggcaggcggtgacttccccactcggggcggagccgcagcctcgcgggggcggggcctggcgccggcggtggcgtcacaaaaggcgggaccacagtggtgtccgagaagtcaggcacgtagctcagcggcggccgcggcgcgtgcgtctgtgcctctgcgcgggtctcctggtccttctgccatcatgccgatgttcatcgtaaacaccaacgtgccccgcgcctccgtgccggacgggttcctctccgagctcacccagcagctggcgcaggccaccggcaagcccccccaggtttgccgggaggggacaggaagaggggggtgcccaccggacgaggggttccgcgctgggagctggggaggcgactcctgaacggagctggggggcggggcggggggaggacggtggctcgggcccgaagtggacgttcggggcccgacgaggtcgctggggcgggctgaccgcgccctttcctcgcagtacatcgcggtgcacgtggtcccggaccagctcatggccttcggcggctccagcgagccgtgcgcgctctgcagcctgcacagcatcggcaagatcggcggcgcgcagaaccgctcctacagcaagctgctgtgcggcctgctggccgagcgcctgcgcatcagcccggacaggtacgcggagtcgcggaggggcgggggaggggcggcggcgcgcggccaggcccgggactgagccacccgctgagtccggcctcctccccccgcagggtctacatcaactattacgacatgaacgcggccaatgtgggctggaacaactccaccttcgcctaagagccgcagggacccacgctgtctgcgctggctccacccgggaacccgccgcacgctgtgttctaggcccgcccaccccaaccttctggtggggagaaataaacggtttagagactaggagtgcctcggggttccttggcttgcgggaggaattggtgcagagccgggacattggggagcgaggtcgggaaacggtgttgggggcgggggtcagggccgggttgctctcctcgaacctgctgttcgggagcccttttgtccagcctgtccctcctacgctcctaacagaggagccccagtgtctttccattctatggcgtacgaagggatgaggagaagttggcactctgccctgggctgcag

SEQ ID NO: 2 - protein, 115 aa, Homo sapiens
MPMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 3 - DNA, 699 bp, Homo sapiens
catccggtgtggtcgacgggtcctccaagagtttggggcgcggaccggagtaccttgcgtgcagttatgtcggcgtcggtagtgtctgtcatttcgcggttcttagaagagtacttgagctccactccgcagcgtctgaagttgctggacgcgtacctgctgtatatactgctgaccggggcgctgcagttcggttactgtctcctcgtggggaccttccccttcaactcttttctctcgggcttcatctcttgtgtggggagtttcatcctagcggtttgcctgagaatacagatcaacccacagaacaaagcggatttccaaggcatctccccagagcgagcctttgctgattttctctttgccagcaccatcctgcaccttgttgtcatgaactttgttggctgaatcattctcatttacttaattgaggagtaggagactaaaagaatgttcactctttgaatttcctggataagagttctggagatggcagcttattggacacatggattttcttcagatttgacacttactgctagctctgctttttatgacaggagaaaagcccagagttcactgtgtgtcagaacaactttctaacaaacatttattaatccagcctctgcctttcattaaatgtaaccttttgctttccaaattaaagaactccatgccactcctcaaaaaaaaaaaaaa

SEQ ID NO: 4 - protein, 113 aa, Homo sapiens
MSASVLSVISRFLEEYLSSTPQRLKLLDAYLLYILLTGALQFGYCLLVGTFPFNSFLSGFISCVGSFILAVCLRIQINPQNKADFQGISPERAFADFLFASTILHLVVMNFVG

SEQ ID NO: 5 - DNA, 1077 bp, Homo sapiens
cttatccctgcgtagaaacgcctgccaatgctttctcatttggacccagactccagatcgggagcagtcttatagctggatcagctaccaagagaagttgtaaaccaagaagagaaaagcatttcaatttgggacatttatttgcacctggaaatggggaatgggctgtcagaccagacttctatcctgtccaacctgccttcatttcagtctttccacattgttattctgggtttggactgtgctggaaagacaacagtcttatacaggctgcagttcaatgaatttgtaaataccgtacctaccaaaggatttaacactgagaaaattaaggtaaccttgggaaattctaaaacagtcacttttcacttctgggatgtaggtggtcaggagaaattaaggccactgtggaagtcatataccagatgcacagatggcattgtatttgttgtggactctgttgatgtcgaaaggatggaagaagccaaaactgaacttcacaaaataactaggatatcagaaaatcagggagtccctgtacttatagttgctaacaaacaagatttgaggaactcattgtcactttcagaaattgagaaattgttagcaatgggtgaactgagctcatcaactccttggcatttgcagcctacctgtgcaatcataggagatggcctaaaggaaggacttgagaaactacatgatatgatcattaaaagaagaaaaatgttgcggcaacagaaaaagaaaagatgaatatcaatacctattatatctgtgtggagtaggttttctctggtctgattttgacaaatagaagagtgtctacaccgtcctttgcctgtctgccctcctggatgctattaaagctttgttttgttgaacaatcagatgcccaactctgttgccttgtggaagatgagtaaatgcagtgcttcttaaagtggtctcttctccctaccccacaaatcttttggtactaccatttggggaagccaagcaaggatagtaaattgaccagaacacagttgtgggaatttggtctgaagttagtgaaataaaactttaaagagtgtc

SEQ ID NO: 6 - protein, 200 aa, Homo sapiens
MGNGLSDQTSILSNLPSFQSFHIVILGLDCAGKTTVLYRLQFNEFVNTVPTKGFNTEKIKVTLGNSKTVTFHFWDVGGQEKLRPLWKSYTRCTDGIVFVVDSVDVERMEEAKTELHKITRISENQGVPVLIVANKQDLRNSLSLSEIEKLLAMGELSSSTPWHLQPTCAIIGDGLKEGLEKLHDMIIKRRKMLRQQKKKR

SEQ ID NO: 7 - DNA, 2379 bp, Homo sapiens
ggaattccggtcggcctctcgcccttcagctacctgtgcgtccctccgtcccgtcccgtcccggggtcaccccggagcctgtccgctatgcggctcctgcctctagccccaggtcggctccggcggggcagcccccgccacctgccctcctgcagcccagcgctgctactgctggtgctgggcggctgcctgggggtcttcggggtggctgcgggaacccggaggcccaacgtggtgctgctcctcacggacgaccaggacgaagtgctcggcggcatgacaccactaaagaaaaccaaagctctcatcggagagatggggatgactttttccagtgcttatgtgccaagtgctctctgctgccccagcagagccagtatcctgacaggaaagtacccacataatcatcacgttgtgaacaacactctggaggggaactgcagtagtaagtcctggcagaagatccaagaaccaaatactttcccagcaattctcagatcaatgtgtggttatcagaccttttttgcagggaaatatttaaatgagtacggagccccagatgcaggtggactagaacacgttcctctgggttggagttactggtatgccttggaaaagaattctaagtattataattacaccctgtctatcaatgggaaggcacggaagcatggtgaaaactatagtgtggactacctgacagatgttttggctaatgtctccttggactttctggactacaagtccaactttgagcccttcttcatgatgatcgccactccagcgcctcattcgccttggacagctgcacctcagtaccagaaggctttccagaatgtctttgcaccaagaaacaagaacttcaacatccatggaacgaacaagcactggttaattaggcaagccaagactccaatgactaattcttcaatacagtttttagataatgcatttaggaaaaggtggcaaactctcctctcagttgatgaccttgtggagaaactggtcaagaggctggagttcactggggagctcaacaacacttacatcttctatacctcagacaatggctatcacacaggacagttttccttgccaatagacaagagacagctgtatgagtttgatatcaaagttccactgttggttcgaggacctgggatcaaaccaaatcagacaagcaagatgctggttgccaacattgacttgggtcctactattttggacattgctggctacgacctaaataagacacagatggatgggatgtccttattgcccattttgagaggtgccagtaacttgacctggcgatcagatgtcctggtggaataccaaggagaaggccgtaacgtcactgacccaacatgcccttccctgagtcctggcgtatctcaatgcttcccagactgtgtatgtgaagatgcttataacaatacctatgcctgtgtgaggacaatgtcagcattgtggaatttgcagtattgcgagtttgatgaccaggaggtgtttgtagaagtctataatctgactgcagacccagaccagatcactaacattgctaaaaccatagacccagagcttttaggaaagatgaactatcggttaatgatgttacagtcctgttctgggccaacctgtcgcactccaggggtttttgaccccggatacaggtttgacccccgtctcatgttcagcaatcgcggcagtgtcaggactcgaagattttccaaacatcttctgtagcgacctcacacagcctctgcagatggatccctgcacgcctctttctgatgaagtgattgtagtaggtgtctgtagctagtcttcaagaccacacctggaagagtttctgggctggctttaagtcctgtttgaaaaagcaacccagtcagctgacttcctcgtgcaatgtgttaaactgtgaactctgcccatgtgtcaggagtggctgtctctggtctcttcctttagctgacaaggacactcctgaggtctttgttctcactgtattttttttatcctggggccacagttcttgattattcctcttgtggttaaagactgaatttgtaaacccattcagataaatggcagtactttaggacacacacaaacacacagatacaccttttgatatgtaagcttgacctaaagtcaaaggacctgtgtagcatttcagattgagcacttcactatcaaaaatactaacatcacatggcttgaagagtaaccatcagagctgaatcatccaagtaagaacaagtaccattgttgattgataagtagagatacattttttatgatgttcatcacagtgtggtaaggttgcaaattcaaaacatgtcacccaagctctgttcatgtttttgtgaattc

SEQ ID NO: 8 - protein, 552 aa, Homo sapiens
MRLLPLAPGRLRRGSPRHLPSCSPALLLLVLGGCLGVFGVAAGTRRPNVVLLLTDDQDEVLGGMTPLKKTKALIGEMGMTFSSAYVPSALCCPSRASILTGKYPHNHHVVNNTLEGNCSSKSWQKIQEPNTFPAILRSMCGYQTFFAGKYLNEYGAPDAGGLEHVPLGWSYWYALEKNSKYYNYTLSINGKARKHGENYSVDYLTDVLANVSLDFLDYKSNFEPFFMMIATPAPHSPWTAAPQYQKAFQNVFAPRNKNFNIHGTNKHWLIRQAKTPMTNSSIQFLDNAFRKRWQTLLSVDDLVEKLVKRLEFTGELNNTYIFYTSDNGYHTGQFSLPIDKRQLYEFDIKVPLLVRGPGIKPNQTSKMLVANIDLGPTILDIAGYDLNKTQMDGMSLLPILRGASNLTWRSDVLVEYQGEGRNVTDPTCPSLSPGVSQCFPDCVCEDAYNNTYACVRTMSALWNLQYCEFDDQEVFVEVYNLTADPDQITNIAKTIDPELLGKMNYRLMMLQSCSGPTCRTPGVFDPGYRFDPRLMFSNRGSVRTRRFSKHLL

SEQ ID NO: 9 - DNA, 3257 bp, Homo sapiens
aacaggcgtgacgccagttctaaacttgaaacaaaacaaaacttcaaagtacaccaaaatagaacctccttaaagcataaatctcacggagggtctcggccgccagtggaaggagccaccgcccccgccccgaccatggccgaggagctggtcttagagaggtgtgatctggagctggagaccaatggccgagaccaccacacggccgacctgtgccgggagaagctggtggtgcgacggggccagcccttctggctgaccctgcactttgagggccgcaactaccaggccagtgtagacagtctcaccttcagtgtcgtgaccggcccagcccctagccaggaggccgggaccaaggcccgttttccactaagagatgctgtggaggagggtgactggacagccaccgtggtggaccagcaagactgcaccctctcgctgcagctcaccaccccggccaacgcccccatcggcctgtatcgcctcagcctggaggcctccactggctaccagggatccagctttgtgctgggccacttcattttgctcttcaacgcctggtgcccagcggatgctgtgtacctggactcggaagaggagcggcaggagtatgtcctcacccagcagggctttatctaccagggctcggccaagttcatcaagaacataccttggaattttgggcagtttcaagatgggatcctagacatctgcctgatccttctagatgtcaaccccaagttcctgaagaacgccggccgtgactgctcccggcgcagcagccccgtctacgtgggccgggtgggtagtggcatggtcaactgcaacgatgaccagggtgtgctgctgggacgctgggacaacaactacggggacggcgtcagccccatgtcctggatcggcagcgtggacatcctgcggcgctggaagaaccacggctgccagcgcgtcaagtatggccagtgctgggtcttcgccgccgtggcctgcacagtgctgaggtgcctaggcatccctacccgcgtcgtgaccaactacaactcggcccatgaccagaacagcaaccttctcatcgagtacttccgcaatgagtttggggagatccagggtgacaagagcgagatgatctggaacttccactgctgggtggagtcgtggatgaccaggccggacctgcagccggggtacgagggctggcaggccctggacccaacgccccaggagaagagcgaaggaacgtactgctgtggcccagttccagttcgtgccatcaaggagggcgacctgagcaccaagtacgatgcgccctttgtctttgcggaggtcaatgccgacgtggtagactggatccagcaggacgatgggtctgtgcacaaatccatcaaccgttccctgatcgttgggctgaagatcagcactaagagcgtgggccgagacgagcgggaggatatcacccacacctacaaatacccagaggggtcctcagaggagagggaggccttcacaagggcgaaccacctgaacaaactggccgagaaggaggagacagggatggccatgcggatccgtgtgggccagagcatgaacatgggcagtgactttgacgtctttgcccacatcaccaacaacaccgctgaggagtacgtctgccgcctcctgctctgtgcccgcaccgtcagctacaatgggatcttggggcccgagtgtggcaccaagtacctgctcaacctaaccctggagcctttctctgagaagagcgttcctctttgcatcctctatgagaaataccgtgactgccttacggagtccaacctcatcaaggtgcgggccctcctcgtggagccagttatcaacagctacctgctggctgagagggacctctacctggagaatccagaaatcaagatccggatccttggggagcccaagcagaaacgcaagctggtggctgaggtgtccctgcagaacccgctccctgtggccctggaaggctgcaccttcactgtggagggggccggcctgactgaggagcagaagacggtggagatcccagaccccgtggaggcaggggaggaagttaaggtgagaatggacctcgtgccgctccacatgggcctccacaagctggtggtgaacttcgagagcgacaagctgaaggctgtgaagggcttccggaatgtcatcattggccccgcctaagggacccctgctcccagcctgctgagagcccccaccttgatcccaatccttatcccaagctagtgagcaaaatatgccccttattgggccccagaccccagggcagggtgggcagcctatgggggctctcggaaatggaatgtgcccctggcccatctcagcctcctgagcctgtgggtccccactcaccccctttgctgtgaggaatgctctgtgccagaaacagtgggagccctgacctgtgctgactggggctggggtgagagaggaaagacctacattccctctcctgcccagatgccctttggaaagccattgaccacccaccatattgtttgatctacttcatagctccttggagcaggcaaaaaagggacagcatgcccttggctggatcaggaatccagctccctagactgcatcccgtacctcttcccatgactgcacccagctccaggggcccttgggacacccagagctgggtggggacagtgataggcccaaggtcccctccacatcccagcagcccaagcttaatagccctccccctcaacctcaccattgtgaagcacctactatgtgctgggtgcctcccacacttgctggggctcacggggcctccaacccatttaatcaccatgggaaactgttgtgggcgctgcttccaggataaggagactgaggcttagagagaggaggcagccccctccacaccagtggcctcgtggttataagcaaggctgggtaatgtgaaggcccaagagcagagtctgggcctctgactctgagtccactgctccatttataaccccagcctgacctgagactgtcgcagaggctgtctggggcctttatcaaaaaaagactcagccaagacaaggaggtagagaggggactgggggactgggagtcagagccctggctgggttcaggtcccacgtctggccagcgactgccttctcctctctgggcctttgtttccttgttggtcagaggagtgattgaacctgctcatctccaaggatcctctccactccatgtttgcaatacacaattcc

SEQ ID NO: 10 - protein, 687 aa, Homo sapiens
MAEELVLERCDLELETNGRDHHTADLCREKLVVRRGQPFWLTLHFEGRNYQASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFQDGILDICLILLDVNPKFLKNAGRDCSRRSSPVYVGRVGSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLTLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLVPLHMGLHKLVVNFESDKLKAVKGFRNVIIGPA

SEQ ID NO: 11 - DNA, 1470 bp, Homo sapiens
gacggtcacccgttgccagctctagcctttaaattcccggctcggggacctccacgcaccgcggctagcgccgacaaccagctagcgtgcaaggcgccgcggctcagcgcgtaccggcgggtttcgaaaccgcagtcctccggcgaccccgaactccgctccggagcctcagccccctggaaagtgatcccggcatcggagagccaagatgccggcccacttgctgcaggacgatatctctagctcctataccaccaccaccaccattacagcgcctcctccaggggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgatgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaacacattcttcattgattggatggccgccctcggtctgacctatgaccggaagaaagtctccaaggccgccatcttggccaggattaaaagaaccggagatggaaactacaagagtggctgagtttggggtccctcaggttcctttttcaaaaaccagccaggcagaggttttaatgtctgtttattaactactgaataatgctaccaggatgctaaagatgatgatgttaacccattccagtacagtattcttttaaaattcaaaagtattgaaagccaaaaaaaaaaaaaaaaaaaaaaaaa

SEQ ID NO: 12 - protein, 359 aa, Homo sapiens
MPAHLLQDDISSSYTTTTTITAPPPGVLQNGGDKLETMPLYLEDDIRPDIKDDIYDPTYKDKEGPSPKVEYVWRNIILMSLLHLGALYGITLIPTCKFYTWLWGVFYYFVSALGITAGAHRLWSHRSYKARLPLRLFLIIANTMAFQNDVYEWARDHRAHHKFSETHADPHNSRRGFFFSHVGWLLVRKHPAVKEKGSTLDLSDLEAEKLVMFQRRYYKPGLLMMCFILPTLVPWYFWGETFQNSVFVATFLRYAVVLNATWLVNSAAHLFGYRPYDKNISPRENILVSLGAVGEGFHNYHHSFPYDYSASEYRWHINFNTFFIDWMAALGLTYDRKKVSKAAILARIKRTGDGNYKSG

SEQ ID NO: 13 - DNA, 1637 bp, Homo sapiens
gaggcgaaccggagcgcggggccgcggtcgccccgaccagagccgggagaccgcagcacccgcagccgcccgcgagcgcgccgaagacagcgcgcaggcgagagcgcgcgggcgggggcgcgcaggccctgcccgccccttccgtccccacccccctccgccctttcctctccccaccttcctctcgcctcccgcgcccccgcaccgggcgcccaccctgtcctcctcctgcgggagcgttgtccgtgttggcggccgcagcgggccgggccggtccggcgggccgggggatggcgctgctggacctggccttggagggaatggccgtcttcgggttcgtcctcttcttggtgctgtggctgatgcatttcatggctatcatctacacccgattacacctcaacaagaaggcaactgacaaacagccttatagcaagctcccaggtgtctctcttctgaaaccactgaaaggggtagatcctaacttaatcaacaacctggaaacattctttgaattggattatcccaaatatgaagtgctcctttgtgtacaagatcatgatgatccagccattgatgtatgtaagaagcttcttggaaaatatccaaatgttgatgctagattgtttataggtggtaaaaaagttggcattaatcctaaaattaataatttaatgccaggatatgaagttgcaaagtatgatcttatatggatttgtgatagtggaataagagtaattccagatacgcttactgacatggtgaatcaaatgacagaaaaagtaggcttggttcacgggctgccttacgtagcagacagacagggctttgctgccaccttagagcaggtatattttggaacttcacatccaagatactatatctctgccaatgtaactggtttcaaatgtgtgacaggaatgtcttgtttaatgagaaaagatgtgttggatcaagcaggaggacttatagcttttgctcagtacattgccgaagattactttatggccaaagcgatagctgaccgaggttggaggtttgcaatgtccactcaagttgcaatgcaaaactctggctcatattcaatttctcagtttcaatccagaatgatcaggtggaccaaactacgaattaacatgcttcctgctacaataatttgtgagccaatttcagaatgctttgttgccagtttaattattggatgggcagcccaccatgtgttcagatgggatattatggtatttttcatgtgtcattgcctggcatggtttatatttgactacattcaactcaggggtgtccagggtggcacactgtgtttttcaaaacttgattatgcagtcgcctggttcatccgcgaatccatgacaatatacatttttttgtctgcattatgggacccaactataagctggagaactggtcgctacagattacgctgtgggggtacagcagaggaaatcctagatgtataactacagctttgtgactgtatataaaggaaaaaagagaagtattataaattatgtttatataaatgcttttaaaaatctaccttctgtagttttatcacatgtatgttttggtatctgttctttaatttatttttgcatggcacttgcatctgtgaaaaaaaa

SEQ ID NO: 14 - protein, 394 aa, Homo sapiens
MALLDLALEGMAVFGFVLFLVLWLMHFMAIIYTRLHLNKKATDKQPYSKLPGVSLLKPLKGVDPNLINNLETFFELDYPKYEVLLCVQDHDDPAIDVCKKLLGKYPNVDARLFIGGKKVGINPKINNLMPGYEVAKYDLIWICDSGIRVIPDTLTDMVNQMTEKVGLVHGLPYVADRQGFAATLEQVYFGTSHPRYYISANVTGFKCVTGMSCLMRKDVLDQAGGLIAFAQYIAEDYFMAKAIADRGWRFAMSTQVAMQNSGSYSISQFQSRMIRWTKLRINMLPATIICEPISECFVASLIIGWAAHHVFRWDIMVFFMCHCLAWFIFDYIQLRGVQGGTLCFSKLDYAVAWFIRESMTIYIFLSALWDPTISWRTGRYRLRCGGTAEEILDV

SEQ ID NO: 15 - DNA, 63 bp, Artificial Sequence
  Description of Artificial Sequence Primer
ggccagtgaattgtaatacgactcactatagggaggcggtttttttttttttttttttttttt

SEQ ID NO: 16 - DNA, 25 bp, Artificial Sequence
  Description of Artificial Sequence Primer
gtcgtcaagatgctaccgttcagga

SEQ ID NO: 17 - DNA, 51 bp, Artificial Sequence
  Description of Artificial Sequence Primer
ggggacaagtttgtacaaaaaagcaggctatgccgatgttcatcgtaaaca

SEQ ID NO: 18 - DNA, 50 bp, Artificial Sequence
  Description of Artificial Sequence Primer
ggggaccactttgtacaagaaagctgggtttaggcgaaggtggagttgtt

SEQ ID NO: 19 - DNA, 32 bp, Artificial Sequence
  Description of Artificial Sequence Primer
aaggattcgggaatgggctgtcagaccagact

SEQ ID NO: 20 - DNA, 31 bp, Artificial Sequence
  Description of Artificial Sequence Primer
ttaagctttcatcttttctttttctgttgcc